(19) United States
(12) Patent Application Publication
(10) Pub. No.: US 2015/0275262 A1
(43) Pub. Date: Oct. 1, 2015

(54) AUTOMATED QUANTIFICATION OF MICROORGANISM GROWTH PARAMETERS THROUGH TEMPORALLY RESOLVED MICROSCOPIC IMAGING

(71) Applicants: Alex Ratushny, (US); David Dilworth, (US); John Aitchison, (US); INSTITUTE FOR SYSTEMS BIOLOGY, Seattle, WA (US)

(72) Inventors: Alex Ratushny, Seattle, WA (US); David Dilworth, Seattle, WA (US); John Aitchison, Seattle, WA (US)

(21) Appl. No.: 14/435,019

(22) PCT Filed: Oct. 11, 2013
(86) PCT No.: PCT/US13/64667

§ 371 Date: Apr. 10, 2015

Related U.S. Application Data
(60) Provisional application No. 61/712,697, filed on Oct. 11, 2012.

(51) Int. Cl. C12Q 1/02

(52) U.S. Cl. CPC C12Q 1/02

(57) ABSTRACT

A method of high-density, multiparameter growth analysis founded upon modeling microcolony expansion on solid media is described. The method extracts the key growth parameters (lag time, doubling time, carrying capacity and viability) that together define microcolony growth from seeded cells. The invention relates to a method to determine growth parameters of cells that is scalable, time-resolved and quantitative.

### TECHNICAL FIELD

The invention relates to a method to determine growth parameters of cells that is scalable, time-resolved and quantitative. More particularly, the invention method relates to time-resolved growth analysis of microcolony expansion on solid media.

### BACKGROUND ART

Growth is a well-established, sensitive metric of cellular fitness that is widely used to interrogate genetic and environmental interactions. The most basic models of microorganism population expansion over time consist of three distinct phases—lag phase, log phase and stationary phase. Each phase is defined by a specific parameter that uniquely contributes to overall fitness. Lag phase, defined by lag time, is the period after initial inoculation wherein little to no growth is observed. Following acclimation, the population enters log phase and expands exponentially at a constant, maximal rate defined by the doubling time. Finally, a rapid cessation of growth is observed as the population enters stationary phase, having reached its maximum attainable level defined by the carrying capacity. By virtue of its linear nature during exponential growth, the log plot of population versus time has classically been employed to extract the three key growth parameters. Lag time is the period up to the attainment of linearity of the log-plot, doubling time is inversely proportional to the slope of the linear region of the log-plot and carrying capacity is the maximum population when the slope of the log plot approaches zero.

In light of its relatively well-understood cell biology and genetic tractability, baker's yeast, Saccharomyces cerevisiae, is a common model organism utilized to elucidate genetic and environmental interactions on a genome-wide scale. Many methods of assessing yeast strain growth characteristics have been described, and most employ liquid culturing. These include direct measurements, such as cell counting and flow cytometry, and indirect measurements, the most common being the turbidity of the growth media measured by absorbance of 600 nm light. Dynamic range limitations associated with many of these methods render them unable to assess all three growth parameters within a single experimental run; thus, analyses are often restricted to only one growth parameter, most commonly doubling time. Furthermore, difficulties associated with maintaining low volume yeast cultures in suspension at high densities limit liquid growth analysis techniques.

The limited quality of liquid culture analysis is also shown in FIGS. 12(a) and (b). FIG. 12(a) refers to saturated (non-growing) yeast cultures serially diluted to known ODs, placed in a BioScreen C™ device and measured over the course of 1 hour. A plot of the growth rate versus initial OD₆₀₀ is shown. Samples with an initial OD of greater than or equal to 1 OD₆₀₀ exhibit negative growth rate caused by settling of cells on the bottom of the BioScreen™ culture apparatus at high cell densities even with maximal shaking. FIG. 12(b) shows typical yeast growth curves (log₂(OD₆₀₀) vs. time) obtained from a BioScreen C™ device. The cell settling artifact can be observed during standard time course experiments as a drop in measured OD₆₀₀ followed by a continued rise until saturation is reached. In the experiment shown, this occurs consistently at about log₂(OD₆₀₀) value of −0.5, regardless of the growth rate of the strain tested (indicating that it is dependent on cell density and not strain growth rate).

The shortcomings inherent to yeast liquid culture analyses have made it commonplace to employ cell spotting as a proxy for strain growth. Cell spotting assays range from patch biofilm analysis, in which a population of cells is delivered as a bolus onto the surface of solid media, to serial dilution analysis, wherein single colonies are obtained. While these methods are universally accepted, there are major caveats to their use. Foremost, despite the demonstration of dynamical growth assessment for populations of cells through the analysis of biofilm intensity on solid media, most large-scale fitness analyses are assessed from a single time point. A lack of temporal resolution makes it impossible to deconvolve the different stages of population growth and, therefore, apparent differences in fitness cannot be unequivocally attributed to the classically defined growth parameters of doubling time, lag time and carrying capacity. In addition, because growth is often assessed at a relatively late stage in population development, when colonies/biofilms are clearly visible to the naked eye and approaching the carrying capacity, edge effects and other local competition artifacts are commonly observed. In the case of the widely used synthetic genetic array (SGA) and epistatic miniarray profile (E-MAP) methods, many of these systematic biases are corrected by the latest generation of analytical tools; however, given that multiple data sets involving many query strains are required to normalize for batch effects, sensitivity is proportional to the scale of the study using these non-dynamical methods, which limits their tactical utility.

### DISCLOSURE OF THE INVENTION

The present invention provides for a scalable platform capable of high-density measurements of strain lag times prior to the attainment of exponential growth, doubling times during exponential growth and carrying capacities at stationary phase through time course imaging of microcolonies growing on solid media. Additional capabilities are demonstrated through the analysis of microcolony shape to detect phenotype switching, and in vivo microcolony fluorescence reporters to detect epigenetic switching. Throughput-enabling improvements such as multiplexing using Quantum Dot cell-wall labeling and the use of line-scanning image acquisition are also described.

The invention thus provides a method for determining the growth rate of cells in a scalable platform that can be applied to high-density measurements of strain lag times prior to the attainment of exponential growth, doubling times during exponential growth and carrying capacities at stationary phase through time course imaging of microcolonies growing on solid media.

Thus, in one aspect, the invention is directed to a method for determining at least one parameter of cells, said growth parameter selected from lag time, doubling time, carrying capacity and viability comprising:

a) spotting or pinning for a plurality of spots, wherein each spot contains between one or a few and thousands of seeded cells in known separated spatial positions onto a spatially unimpeded solid growth medium on a transparent support;

b) allowing the cells in each spot to grow into a microcolony, where each microcolony develops from said single cell (multiplicity of seeded cells);

c) obtaining periodic images of said microcolonies by high resolution optical detection; and
d) analyzing the images of said microcolonies within each of said spot to determine the size of said microcolonies as a function of time, thus determining lag time, doubling time and/or carrying capacity.

The method of the invention also includes analyzing the shape of the microcolonies within each spatial position, wherein the shape can be correlated with phenotype.

In some embodiments, the cells are fluorescently labeled and the images include detection of in vivo fluorescently-tagged reporter proteins by high resolution optical detection; wherein the fluorescence intensity within each spatial position is analyzed and the intensity of the colony over time represents the gene expression. The dynamic distribution of fluorescence signal can be mapped within spatial positions as a means of monitoring epigenetic switching in developing microcolonies in real-time.

Thus, the invention method can be used to determine gene expression, and, by varying the medium, for example, determine the effect of growth conditions on said gene expression.

Since a multiplicity of individual microcolonies are analyzed all at once, each of which results from only a few cells from an original cell sample, by comparing the growth parameters among the various multiplicity of microcolonies, heterogeneity of an original cell sample can be determined.

In another aspect, the invention is directed to a method for determining the effect of a test compound or composition including environmental factors and medium components on cell growth, comprising:

conducting the method of claim 1 in the presence and absence of said compound or composition; and comparing the parameters measured in the presence and absence of said compound or composition;

wherein a difference in said parameters is indicative of an effect on cell growth of the compound or composition.

### BRIEF DESCRIPTION OF THE DRAWINGS

FIG. 1(a) shows the ODELAY method wherein the four stages of solid-phase growth parameter derivation are described. FIG. 1(b) shows hemispheric modeling of microcolony expansion and relevant calculation.

FIGS. 2(a)-2(g) depict the results of hemispheric modeling of microcolony expansion on solid media.

FIGS. 3(a)-3(g) illustrate the comparison of ODELAY and OD₆₀₀ calculated doubling times.

FIGS. 4(a)-4(f) show the determination of lag time by ODELAY.

FIGS. 5(a)-5(m) illustrate the multiparameter ODELAY analysis of yeast null mutants.

FIGS. 6(a)-6(e) show the complex fitness phenotypes revealed by ODELAY analysis.

FIGS. 7(a)-7(b) depict slow-growing strains selected for multiparameter ODELAY analysis.

FIGS. 8(a)-8(b) illustrate automated ODELAY analysis I. Raw image data to CellProfileOutput.

FIG. 9 illustrates automated ODELAY analysis II, from CellProfilerOut to Growth Parameter Extraction.

FIGS. 10(a)-10(g) illustrate the Automated ODELAY analysis III, showing growth parameter extraction from modeled growth curves.

FIGS. 11(a)-11(d) depict the Automated ODELAY analysis IV, giving results for various strains in comparison to manual analysis.

FIGS. 12(a)-12(b) show a prior art method to obtain growth curves for yeast.

### MODES OF CARRYING OUT THE INVENTION

The method of the invention is denoted ODELAY (One-cell Doubling Evaluation by Living Arrays of Yeast, although it applies to cell growth in general). It provides for analysis, including multiparameter analysis, of growth kinetics that is founded on microscopic time-course imaging of cells growing on solid media to form microcolonies from one or a few cells.

ODELAY is applicable to a wide range of growth substrates and incubation temperatures and is highly scalable, as it has the potential to analyze multiple individual microcolonies for up to thousands of different cells on a single microscope slide using DNA microarray pinning technologies.

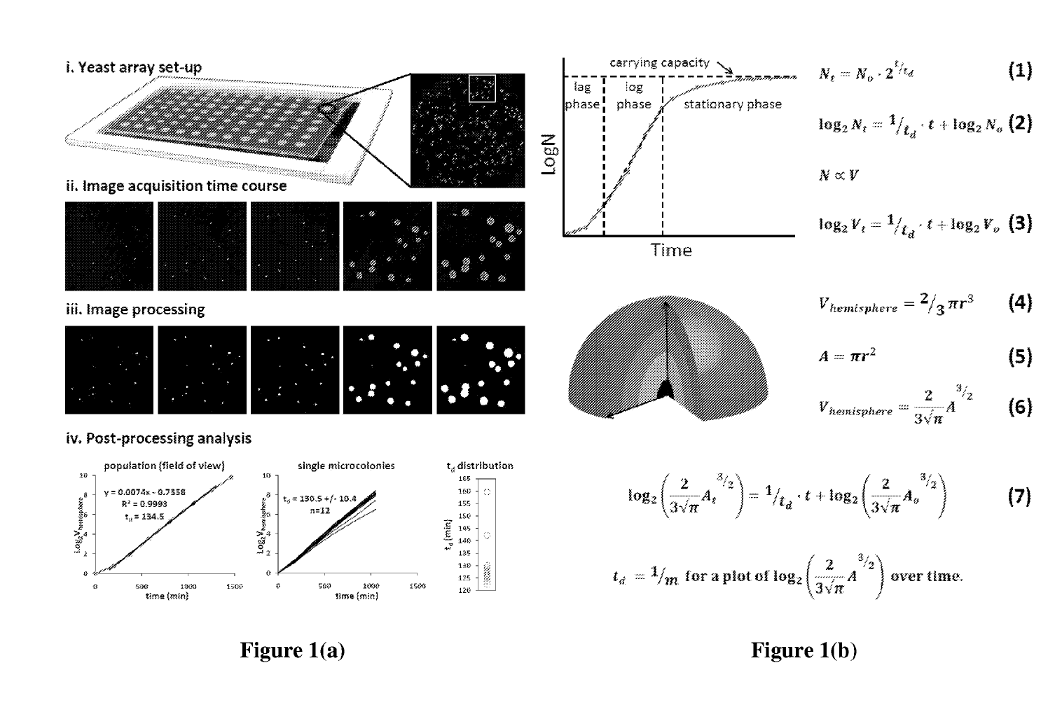

ODELAY is depicted schematically in FIG. 1 and consists of four stages: (i) spotting or pinning of ordered arrays of live cells onto thin beds of growth substrate on a glass slide support; (ii) periodic bright field image acquisition over a user-specified time course; (iii) processing of raw bright field data to extract microcolony cross-sectional area data and (iv) post-processing calculation of growth parameters for each sample as a population and for individual microcolonies within each sample.

Key growth parameters (lag time, doubling time, carrying capacity and seed population viability) that together define the fitness of cells under different genetic or environmental contexts can be quantified by this method. Growth parameter determination is achieved by taking advantage of the correlation between cell population and cross-sectional area of developing microcolonies as they as they expand from single cells. Growth curves for each individual microcolony are generated based on cross-sectional area information, followed by modeling of a Gompertz function to the experimental data and, finally, calculation of the key growth parameters for each microcolony, for example, by derivative analysis of each fitted Gompertz function.

The straightforward property of viability may be of sufficient interest in some cases to take advantage of the ability of the invention method to evaluate numerous microcolonies at one time wherein precise numerical values of the other growth parameters (lag time, doubling time and carrying capacity) are not necessary. For example, if the effectiveness of a drug to be evaluated can be judged simply based on the ability of the drug to influence the viability of cells, measurement of this parameter may be sufficient. Similarly, the effect of environmental factors which may be toxic to cells can be evaluated using this simple criterion. However, the method of the invention, by providing for periodic imaging of cell growth, permits quantitative evaluation of the three major growth parameters.

The evaluation of these parameters is based on the size of the microcolonies as a function of time. The size is a measure of the number of cells in a colony, based on measurement of physical dimensions. The volume occupied by the colony is a measure of its size or number of cells and this volume can be estimated according to the method of the invention based on the cross-sectional area by assuming a hemispherical cap on the cross-sectional area for yeast cells as further described below. For other types of cells, volume measurements assume other shapes that can be readily calculated based on the assumed shapes. If only relative growth is to be measured, a good approximation may be obtained using cross-sectional area alone. Furthermore, many bacteria spread in a single-cell laminar sheet and in this case, as well, just the cross-sectional area is a direct measurement of population.

For relative measurements, cross-sectional area can be employed because it is assumed that cells of all strains of a given organism form microcolonies that have the same cross-sectional area:volume ratio.

As defined herein, a colony is a mass of cells detectable by the naked eye that result from seeding with a single cell. It is distinguished from a “microcolony” qualitatively by the naked eye threshold of detection. A “microcolony” is the result of the developmental period from a single or a few cells up to several thousand cells. Because each microcolony is seeded from one to a few cells and many microcolonies can be analyzed for each set of cells, the heterogeneity of the quantitative growth parameters can be assessed on a cell by cell basis, as can the viability of each seed population. Through increased sensitivity and the potential for growth parameter profiling, the enhanced resolution afforded by the invention method of multiparameter fitness assessment can facilitate the generation and/or refinement of gene-gene and gene-environment interaction networks for any microcolony-forming organism or cell.

The number of microcolonies to be evaluated in a single experiment varies according to the needs of the researcher. If desired, as few as 10 microcolonies per transparent support could be evaluated in up to hundreds of transparent supports. Typically 500 or 1,000 or more microcolonies can be evaluated using 100 spots per transparent support, e.g., and multiple transparent supports giving, for example, essentially simultaneously, up to 100,000 or more growth curves per experimental run. In this enumeration, as throughout the specification herein, indications of various boundaries of a range are meant to include individual integers within that range. Thus, here, by mentioning 10 or 25, the statement should be read to include 11, 12, . . . 24, etc.

The importance of this methodology is at least two-fold: (1) because analyzed microcolonies derive from single or only a few seeded cells, the ODELAY method has a very high measurement density potential (hundreds per mm²) sometimes referred to as “Lab-on-a-chip” technique. (2) The ability to obtain a large number of growth parameter measurements for each microcolony by ODELAY permits the study of population heterogeneity at the single cell level.

The addition of temporal resolution to growth analysis at a throughput that is amenable to genome-wide studies is an important feature of this method. Traditionally, genome-wide studies employ single time point ‘fitness’ assessment. ODELAY achieves a sensitivity and resolution unattainable by traditional methods because, unlike single time point analyses, ODELAY is able to deconvolve the contributions of each growth parameter to the overall growth kinetics of each microcolony.

Most approaches to converting the raw image data as a quantitative measure of growth of cells focused on the macrocolony scale have suffered from a low measurement density, and have demonstrated that, when visible to the naked eye, it is the colony's integrated density, not its cross-sectional area, that is most correlated with population.

The method of the invention is applied to cell types that can be imaged and detected against background signal by automated edge detection algorithms. These cells are derived from single-celled and multicellular organisms including, for example, yeast, bacteria, archaea, fungi, protozoa, animal (e.g., mammalian, avian and insect) and plant cells. To date, this method has been successfully applied to yeast (Saccharomyces cerevisiae), halobacteria (Halobacterium salinarum) and cyanobacteria (Synechococcus elongatus). Also suitable are human disease microorganisms, including Mycobacterium tuberculosis, the organism responsible for tuberculosis.

This method assesses and measures growth rate of single or a few cells or up to thousands of cells into microcolonies in a spatial array on a solid growth medium. The solid growth medium is plated/coated on a transparent support such as glass, Plexiglas®, or other polymers or quartz. By a “transparent” support is meant a sufficient transparency to allow optical measurement. The support and its medium permit unimpeded growth, which means that there is no cover slip or other barrier that prevents the cells from growing in three dimensions.

The type of growth medium will be dependent on the type of cell selected. Types of growth media are those appropriate for the cells to be used in the method. The growth medium may be the same or different across the field of cells. Thus, although the medium may be uniform throughout the support for all of the various spots, each individual spot may have a different medium or a gradient can be introduced across the support with respect to various components of the medium. Various methods are available for applying media to individual spots, such as inkjet application.

Because the medium can be varied among the multiplicity of cells imaged, the effect of various media on growth parameters can be measured, especially helpful when similar cells are used in the spots to be compared. Alternatively, the heterogeneity of an original cell sample can be determined by taking one or a few cells from the sample to seed each spot and comparing the growth parameters including viability among the various spots. The growth medium may further comprise magnetic particles in a predetermined arrangement on the surface of the medium to allow accurate automatic focusing.

The cells may be labeled or tagged with color/fluorescent dye or other optically-determinable tags to aid identification in multiplexed experiments (experiments involving multiple strains within a single region of interest and analysis of gene expression simultaneously with growth rate. For example, a fluorescent protein may be coupled to control sequences that effect expression of genes that dependably produce protein in a uniform manner throughout the growth cycle. Alternatively, quantum dots may be attached to the cell wall to give multiplexed color combinations to multiplex different strains within a single experiment. Methods for quantum dot multiplexation of yeast using cell wall labeling are found on the World Wide Web at ncbi.nlm.nih.gov/pubmed/20694809. Multiplexing employs various combinations of such QDOTs. Similarly, fluorescent proteins of various colors can also be used to permit multiplexing, e.g., combinations of GFP, YFP, CFP, RFP and/or BFP can be used.

In addition to the measurement of growth parameters or viability, the cells seeded in each spot may be provided with reporter proteins for gene expression that are coupled to measurable tags, such as fluorescent moieties, or the measurable tags can be placed under control of promoters that are associated with genes of interest. By measuring the fluorescence intensity as a function of time, a record of gene expression is thus also obtained. Comparisons may also be made among the various spots to assess differences in gene expression patterns. Such reporting systems may be multiplexed using various colors of fluorescent protein to monitor expression levels of multiple genes.

The cells are placed in a spatial array on the growth medium in known spatial positions by spotting or pinning techniques such as DNA microarray pinning technologies. See FIG. 1 for an example of spotting. Pinning using DNA microarray pins is well known in the art and pins designed for DNA microarray production can spot arrays of yeast, for example, at ˜8 k spots per surface substrate. The volume deposited by a given pin is dependent on the area of the pinhead that contacts the surface. A 600 μm pin deposits ˜12.5 nL ˜125 cells/spot @ 1×10⁷ cells/mL.

The cells that are seeded in each spot may be derived from a single sample of cells and tested for heterogeneity or for response to a variety of growth conditions, environmental factors or for response to a multiplicity of drugs or other compounds or compositions of interest. The spots may also be seeded with different types of cells that may differ in as little as a single allele or may be derived from entirely different species or genera. The design of a particular application of the method will vary according to the types of cells employed, the media emp^{l}oyed in each spot, the presence or absence of test environmental substances, and the like, depending on the interest of the practitioner.

The growth of the cells is determined by obtaining periodic images with high resolution optical detectors, such as standard CCD-cameras. “Periodic” images refer to images taken with sufficient repetition to measure growth within the desired growth stage. Depending on the proliferation rate of the cells, the images can be acquired during a time course of a little as 4-6 hours, or 5-10 hours or 8-12 hours or 10-24 hours or over several days or can extend to up to two or three weeks or longer. The size of microcolonies determined from the periodic images within each spatial position can be used to determine the growth parameters. For yeast, microcolony volume may be calculated from the cross-sectional area modeled to the base of a hemisphere. Volume of organisms that do not grow hemispherically can be calculated from cross-section area in other ways.

The growth parameter of the cells is determined by obtaining periodic images during exponential/logarithmic (log phase), lag phase or the stationary phase of cell growth. The periodic images at intervals of 5 minutes-3 hours depending on the growth rate. Uniform intervals are not necessarily required, but are often convenient. Thus, intervals of 5 minutes, 10 minutes, 20 minutes, 30 minutes, 45 minutes, one or several hours and any arbitrarily chosen period suitable for the growth rate of the cells is chosen. For slower growing microorganisms, the total time course will be significantly longer than for faster-growing cells (a week or more) due to the time required to attain carrying capacity and the sampling interval can be significantly longer as well (as little as once or twice a day) because oversampling is not necessary. To assess the distribution of lag times the method may further include obtaining image at time zero, i.e., at the start of the experiment. The starting point for obtaining the periodic images will depend on the growth parameter it is desired to measure. Similarly, if gene expression levels are measured, the interval between images and the starting point will depend on the nature of the information the practitioner desires to derive.

“Cellular fitness” can also be evaluated. Essentially, this is a measure of the competitive advantage of one strain as compared to another. If two different strains of microorganism or cells from multicellular organisms are grown in the same environment for a period of time, the relative numbers of cells in each population can be compared. This is a more nuanced measure of growth since there may be variations in growth rate in lag time that differ from those, for example, in exponential phase. The growth rate used for comparison is generally based on biomass but the biomass will accumulate at differing rates during the various phases of growth. Thus, the growth curves determined must take account of these differences.

The invention may also be used to test the effect of, for example, a drug (or other environmental factor) on growth. Thus, the method may be used for determining the effect of a compound or composition, by contacting a compound or composition with the cells that are subject to the invention method and measuring the various parameters of the growth rate of the cells. The effect is analyzed by comparing these parameters of the cells subjected to the compound of composition to that of cells not contacted with the compound or composition, whereby a difference in one or more of these parameters is indicative of an effect of the compound or composition. This analysis may be conducted as part of a screening process to identify drug candidates. Other compounds or compositions of interest represent environmental pollutants, toxins, growth factors, and the like.

Thus, the methods of the invention have various applications. Basically, the methods can quantitatively obtain growth parameters and determine the time course of gene expression. These may be of interest per se as measures of cell heterogeneity in a sample, or as measures of differences between organisms from which the cells are derived. The results are also of interest by virtue of their dependence on factors external to the cells that influence the results. These external factors include environmental conditions such as temperature, components of the growth medium designed for influencing said growth, components of the medium that are added to test their effect, per se, such as toxins, environmental pollutants, and drugs. The design of the spatial array and the medium as well as the time course of measurement is dependent on the nature of the experimenter's interest and the design of the particular cells used, media used, and the like, and the parameters to be measured are within the capabilities of the ordinary practitioner.

The following example is offered to illustrate but not to limit the invention.

### Example

### Methods

Yeast Strains and Growth Conditions.

Unless otherwise specified, all experiments were performed at room temperature (23+/−3° C.) using rich growth media, YEPD, [1% w/v yeast extract (BD), 2% w/v peptone (BD), 2% w/v dextrose (BDH)]. Galactose growth media contained 2% w/v galactose (Acros) in place of glucose and solid media contained 2% w/v agar (BD) for cell spotting assays or 1.0% w/v agarose (Invitrogen) for ODELAY analyses. S. cerevisiae strains used in this study are listed in Table 1.

TABLE 1
Yeast strains utilized in this study

| Strain | Genotype | Source |
|---|---|---|
| BY4741 | MATa hisSd1 leu2d0 met15d0 ura3d0 | ¥ |
| BY4742 | MATa his3d1 leu2d0 lys2d0 ura3d0 | ¥ |
| M316/1A | prp20-7 MATa ade2-101 his3d200 tyr1 ura3-52 | # |
| ΔRAI1 | YGL246C::KANr MATa his3d1 leu2d0 lys2d0 ura3d0 | ¥ |
| ΔYBR267W | YBR267W::KANr MATa his3d1 leu2d0 met15d0 ura3d0 | ¥ |
| ΔYDL013W | YDL013W::KANr MATa hid3d1 leu2d0 met15d0 ura3d0 | ¥ |
| ΔYDL033C | YDL033C::KANr MATa his3d1 leu2d0 met15d0 ura3d0 | ¥ |
| ΔYDL083C | YDL083C::KANr MATa his3d1 leu2d0 met15d0 ura3d0 | ¥ |
| ΔYCR063W | YCR063W::KANr MATa his3d1 leu2d0 met15d0 ura3d0 | ¥ |
| ΔYDL074C | YDL074C::KANr MATa his3d1 leu2d0 met15d0 ura3d0 | ¥ |
| ΔYCR071C | YCR071C::KANr MATa his3d1 leu2d0 met15d0 ura3d0 | ¥ |
| YCL032W | YCL032W::KANr MATa his3A1 leu2A0 met15d0 ura3d0 | ¥ |
| YBR267Wα | YBR267W::KANr MATa his3d1 leu2d0 lys2d0 ura3d0 | ¥ |
| 3-1 Wild-type | n his3d1 leu2d0 ura3d0 | § |
| 3-2 d YBR267W | YBR267W::KAN^{r} YDL033C::KAN^{r} n his3d1 leu2d0 ura3d0 | § |
| AYDL033C | | |
| 3-3 AYBR267W | YBR267W::KAN^{r} n his3d1 leu2d0 ura3d0 | § |
| 3-4 AYDL033C | YDL033C::KAN^{r} n his3d1 Ieu2d0 ura3d0 | § |

¥ Winzeler, E. et al. Functional Characterization of the Saccharomyces cerevisiae Genome by Gene Deletion and Parallel Analysis. Science. 285: 901-906. (1999)
# Amberg, D. C. et al. Nuclear PRP20 protein is required for mRNA export. EMBO J. 12: 233-241. (1993)
§ This study. Haploid sister spores. Segregation of MAT, MET15 and LYS2 alleles undetermined.

All strains have been previously described with the exception of strains used to test for ODELAY phenotypes resulting from combinatorial effects of individual mutants, which were generated as follows from a MATαΔBR267W strain and a MATαΔYDL033C strain in the BY4742 and BY4741 strain backgrounds, respectively. Exponentially growing liquid cultures of the two strains were mixed 1:1 and cells were allowed to mate for 6 hours at room temperature without agitation of the media after which a small amount of the resuspended liquid culture was transferred to a YPD plate. Nascent zygotes, identified visually by their characteristic barbell shape, were transferred to isolated areas of the YPD plate using a microdissection needle and colonies were allowed to form by growth at 30° C. for 2 days. A heterozygous diploid was sporulated and spores were manually microdissected by standard methods. After initial screening for tetrads that exhibited G418 resistance in 3 of 4 sister spores, tetrads segregating 1:1:1:1 for the genotypic combinations of ΔYBR267W and ΔYDL033C were identified by gene-specific PCR.

Growth Rate Determination by OD₆₀₀.

Manual optical density measurements of yeast cultures were obtained using a 1 cm path BioPhotometer™ spectrophotometer (Eppendorf). For early time points, when cell density lies below or within the linear range of instrument (0.1<OD₆₀₀<0.4), samples were read directly and, for later time points, cultures were diluted appropriately to obtain OD₆₀₀ readings within this range.

BioScreen™ Doubling Time Determination.

Automated optical density measurements of yeast cultures were obtained using a BioScreen C™ (Growth Curves USA) using manufacturer's suggested protocols with the exception that culture volume was reduced to 200 μL to prevent artifacts arising from liquid splashing onto the plate lid during maximal agitation and a starting OD₆₀₀ of 0.01 was utilized in order to ensure that cultures were in exponential phase once they entered the empirically determined linear range of the instrument.

Manual ODELAY. Slide Preparation and Yeast Array Setup.

Growth media was prepared as a 1:1:8 mixture of the following sterile stock solutions, respectively, 10×YEP (10% w/v yeast extract and 20% w/v peptone), 20% w/v carbon source (glucose or galactose) and 1.25% w/v agarose in water. Typically, a 100 mL volume of 1.25% agarose stock was prepared, divided into 16 mL aliquots in 50 mL conical bottom tubes and stored at 4° C. until use. Aliquots were placed in rapidly boiling water for 10-15 minutes to completed melt the agarose gel and, once molten, 2 mL each of 10×YEP and 20% carbon source were added, yielding a final growth substrate containing 1% yeast extract, 2% peptone, 2% carbon source and 1% agarose. Heating was continued for an additional 5 minutes and then the molten solution was passed through an Acrodisc® 1.2 μm Versapor® membrane syringe filter (PALL Life Sciences) into 1 mm or 2 mm thickness SDS-PAGE glass cassette (BioRad) preheated to 90° C. The apparatus was allowed to cool to room temperature and, after careful separation of the glass plates, slabs of solid media were cut into size and transferred to microscope slides. Slides were equilibrated overnight in a humid chamber and the following day yeast in liquid culture, diluted to an OD₆₀₀ of ˜0.05, were spotted onto agarose slabs using a 384 pin manual pinning device, a multichannel pipet or a Matrix® Hydra DT fluidics robot (ThermoScientific). Slides were air dried for 5 minutes and then stored in an enclosed, humidified chamber.

Image Acquisition Time Course.

Bright field images were periodically captured using a 4× objective with an Eclipse TS100 microscope (Nikon) equipped with a DFC295 digital camera (Leica) and Application Suite v3 (Leica) image acquisition software. Panoramas of spots containing 100 to 300 cells/microcolonies were compiled from multiple overlapping images with an automated stitching plug-in (Preibisch, S., et al., Bioinformatics (2009) 25:1463-1465) written for the open-source Image J-based application, FIJI (located on the World Wide Web at pacific.mpi-cbg.de/wiki/index.php/Fiji) or stitched manually in cases where automated stitching failed. To aid in automated stitching, images were first subjected to global illumination correction using CellProfiler v10211 (located on the World Wide Web at cellprofiler.org) and, after stitching, fields of view containing approximately 50 cells in regions faithfully stitched throughout the time course were extracted for further analysis.

Image Processing.

Time course images were subjected to semi-automated edge-finding, inversion and binarization with a Photoshop (Adobe) action that thresholds based on the selection of background pixels with the Magic Wand Tool at an empirically-derived tolerance.

Post Processing Analysis.

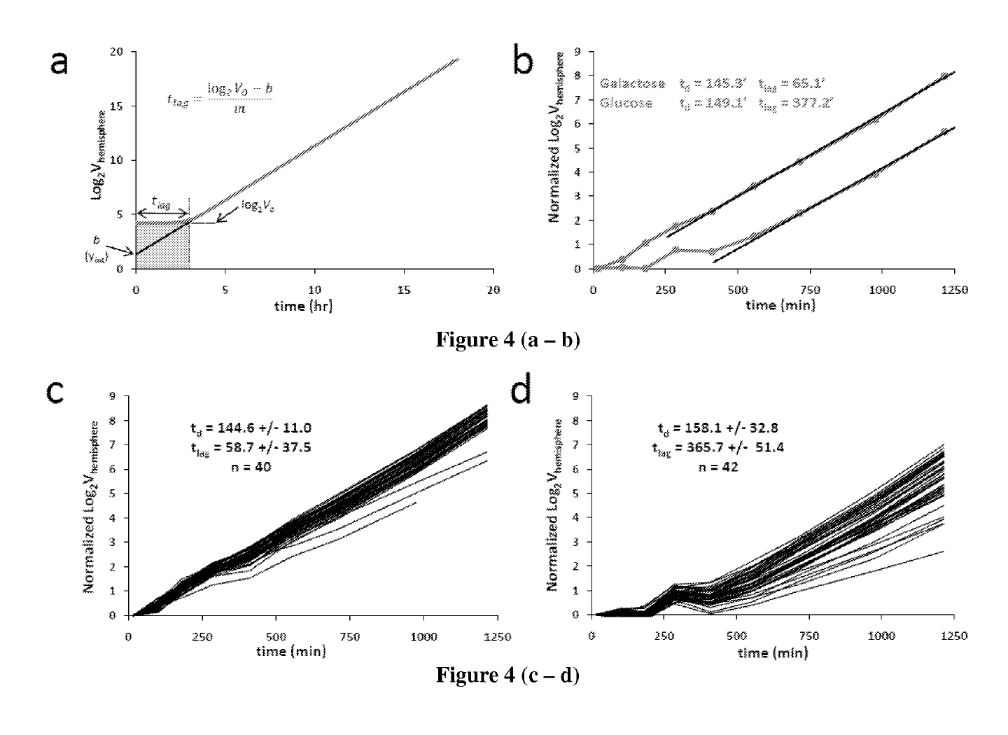

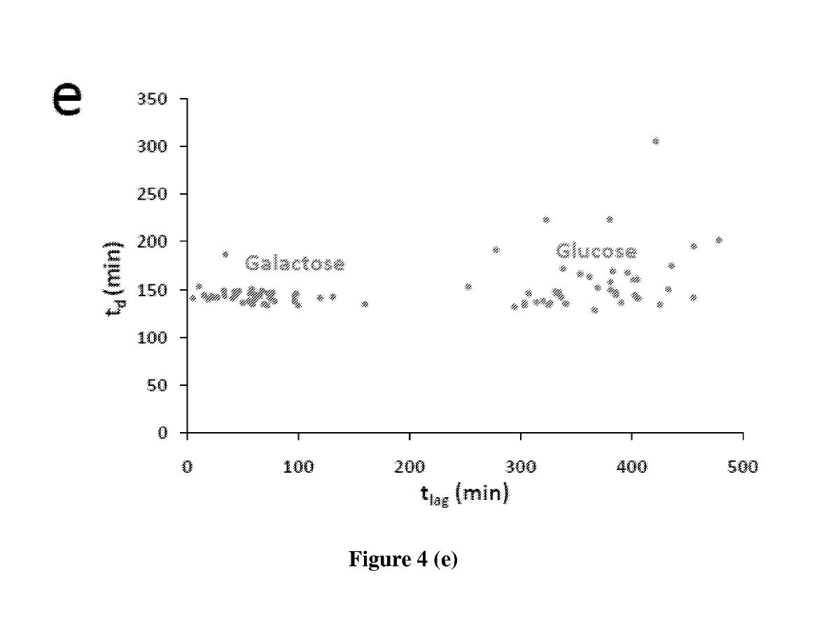

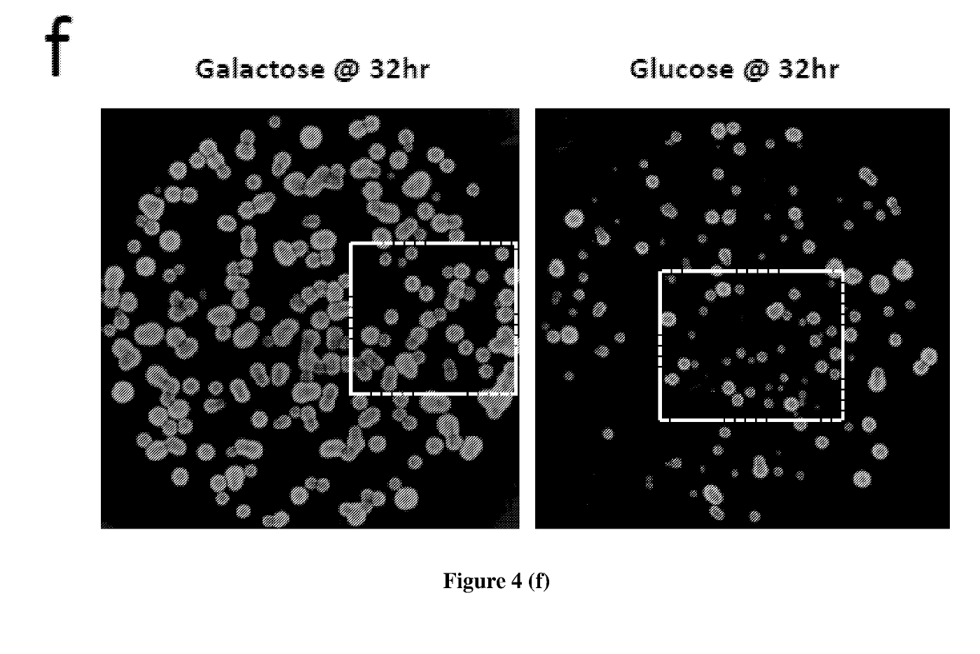

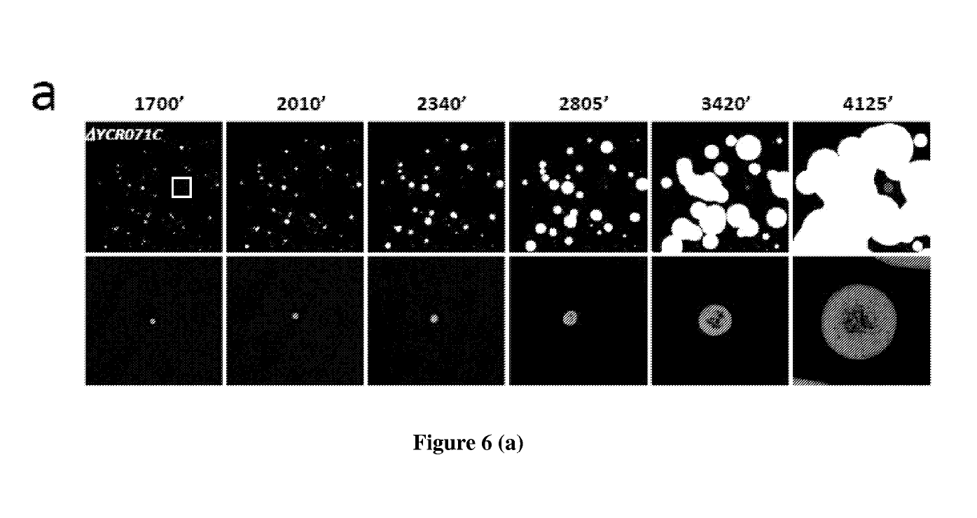

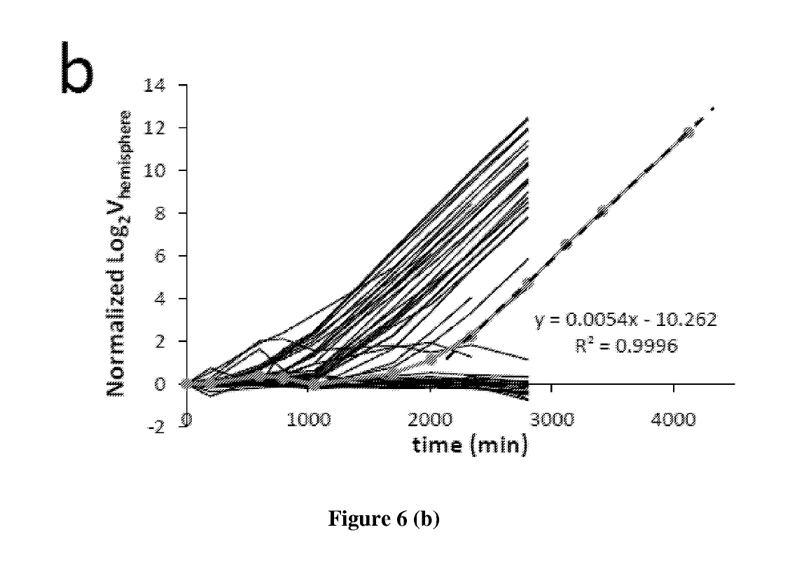

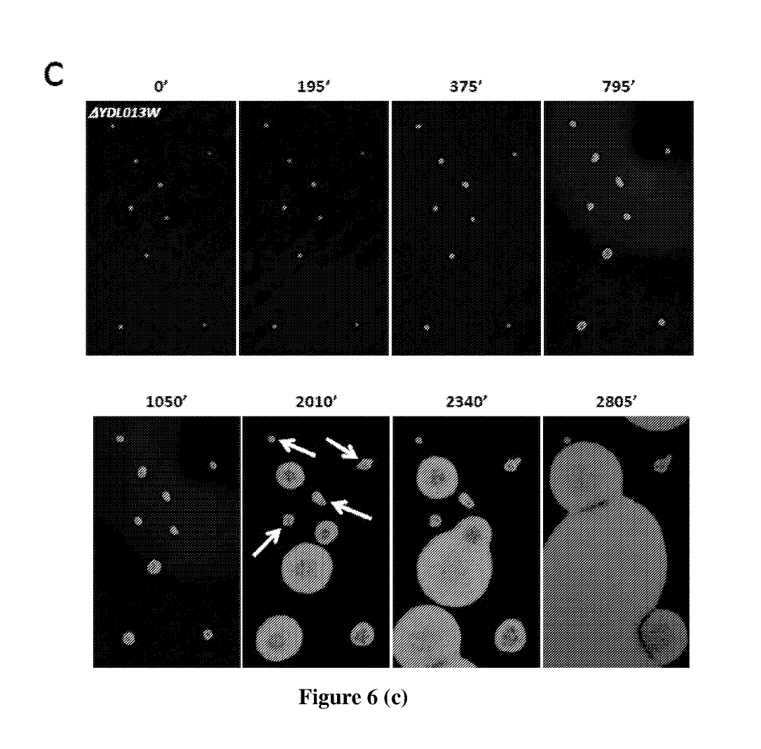

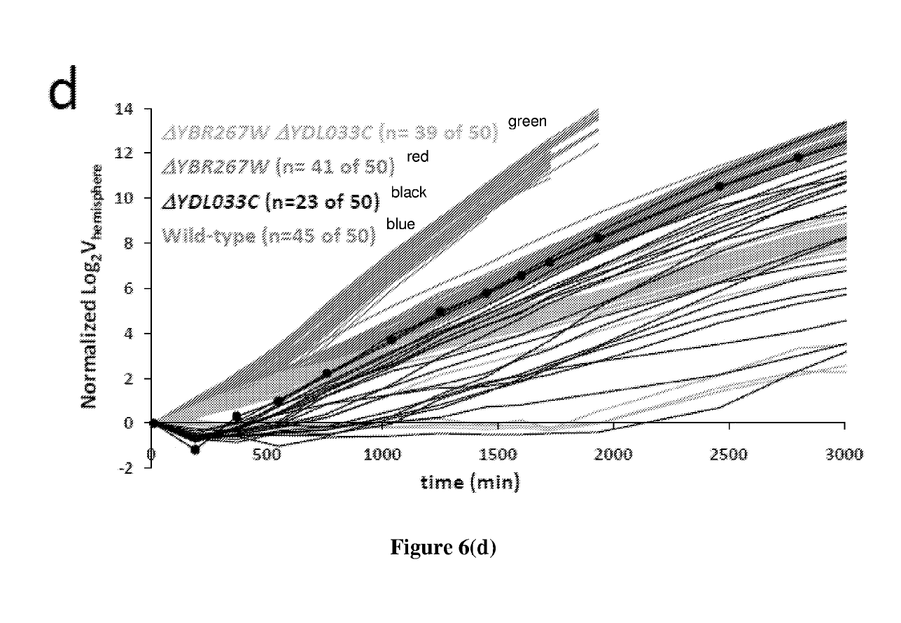

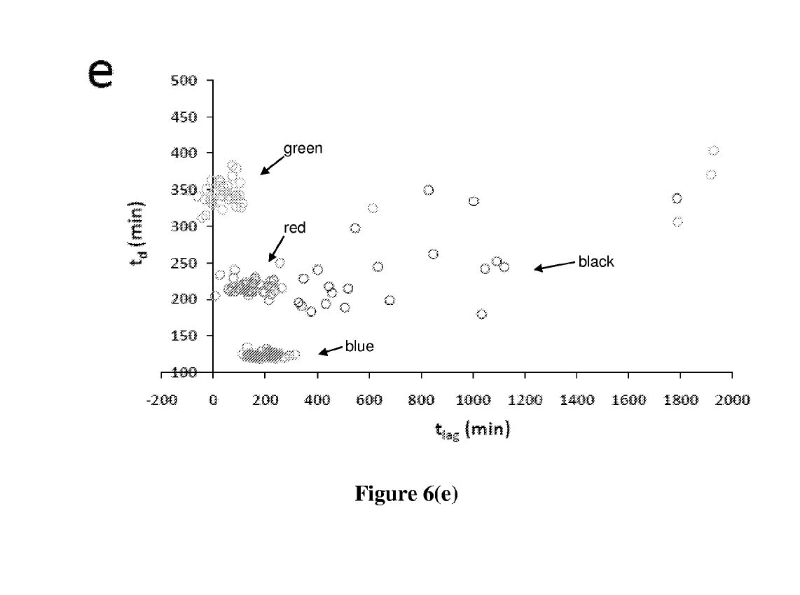

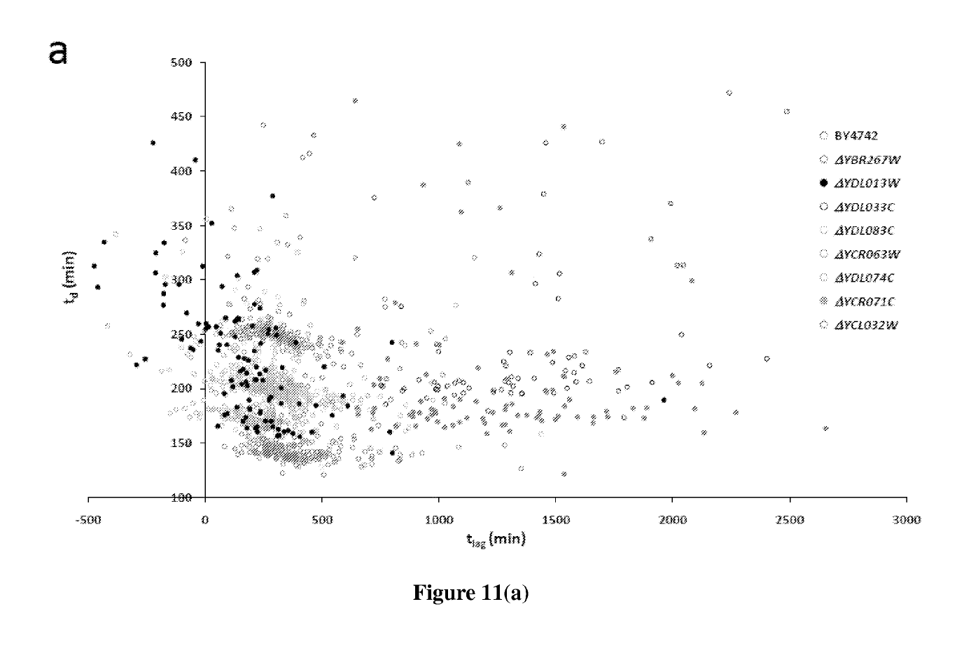

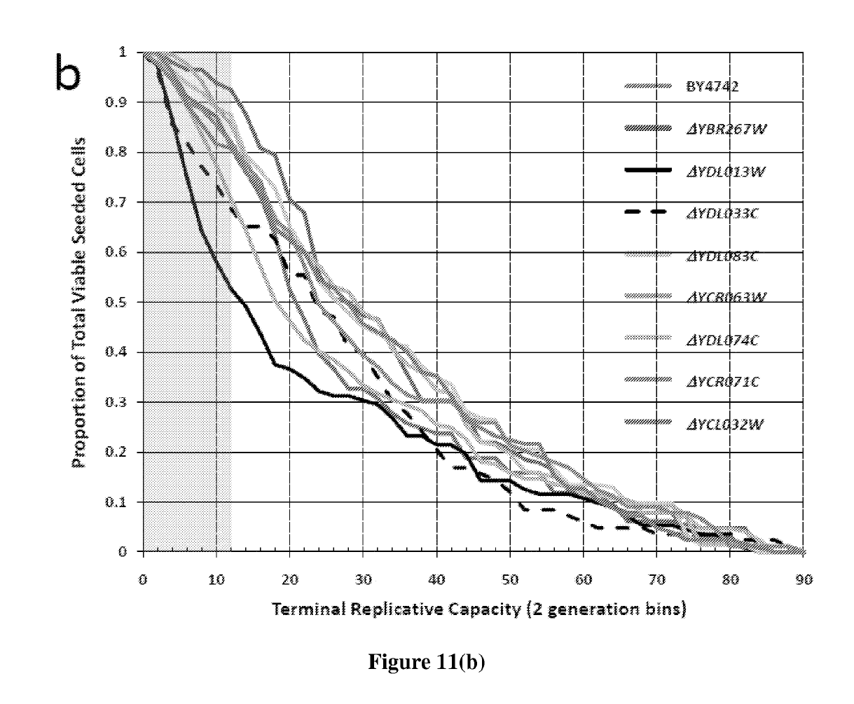

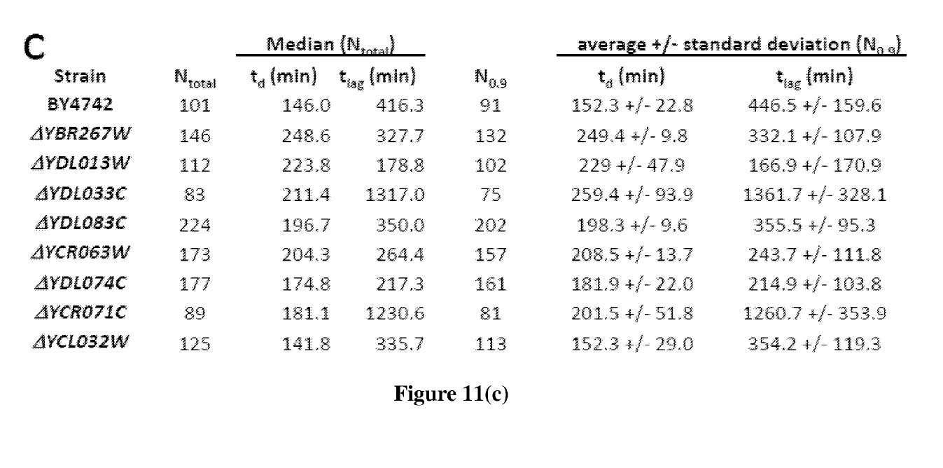

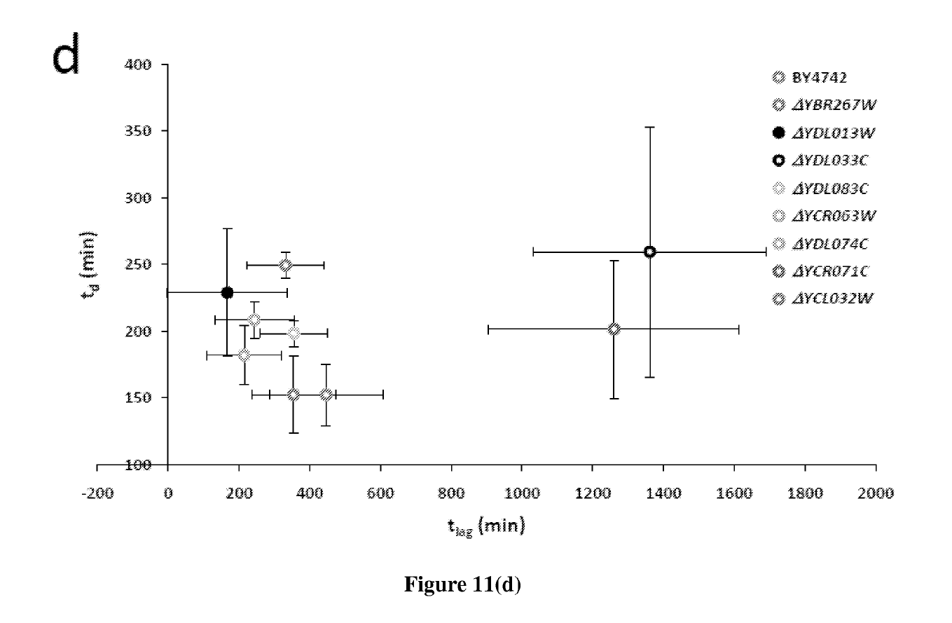

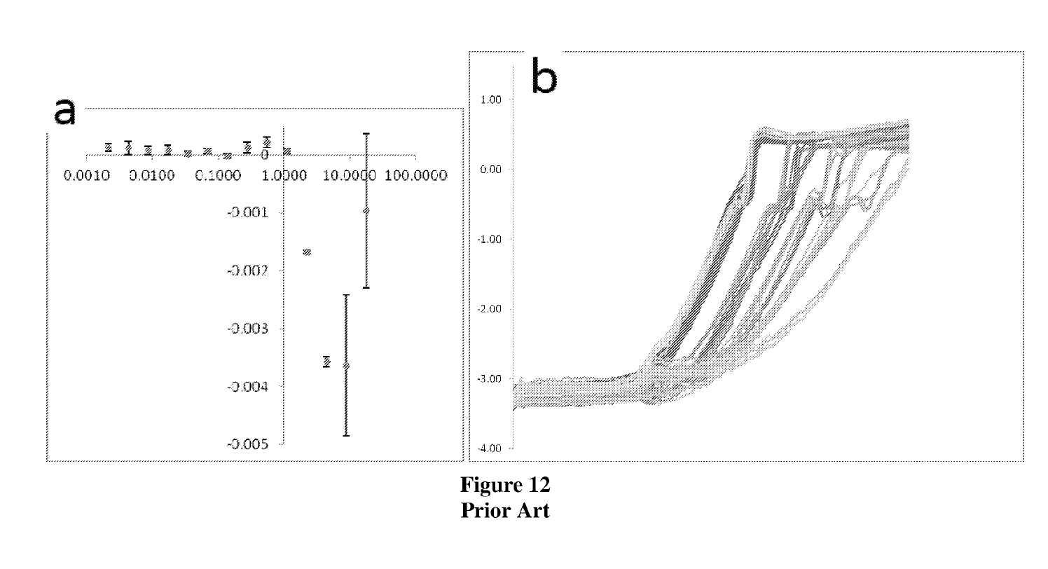

Cross-sectional area data were extracted from manually identified and tracked objects (cells or microcolonies) within binarized images with FIJI using integrated density measurement. Data were compiled, analyzed and prepared for presentation in Excel™ (Microsoft). Microcolony volume was calculated based on cross-sectional area measurements using Eq6 (FIG. 1(b)) and the log₂ of hemispheric volume was plotted versus time. User-defined windows of exponential growth were employed for best-fit line calculations from which doubling times were obtained as the inverse of the slope of the best-fit line. Lag time was calculated from the log₂ value of the initial cell/microcolony volume (Vₒ) and the parameters of the best fit line equation describing the region of exponential growth, as shown in FIG. 4(a). For the best fit line equation, y=mx+b, where the slope (m) and y-intercept (b) are known, the lag time, t_{lag}, is the value of x when the value of y is set to log₂Vₒ, the base-2 log of the initial hemispheric volume. For individual microcolony analyses, strain lag times and doubling times are expressed as average+/−standard deviation after elimination of data derived from microcolonies that exhibited no growth or marked decrease in growth rate over the time course. This was achieved by limiting data to include only microcolonies for which 100<t_{d}<500 minutes and −100<t_{lag}<2000 minutes. A negative lag time was permitted in this cut-off to accommodate strains with short lag times for which lag time distributions were observed to extend into this region (see FIG. 6(e) and FIG. 11(a)).

Automated ODELAY.

Slide preparation and yeast array set-up are performed as described above for manual ODELAY. Image acquisition and panorama stitching can also be performed as described for manual analysis; however automation of these steps has been achieved using a DeltaVision personal DV microscope (Applied Precision) and this, or a similarly capable system, is recommended for larger samples sizes. Automated ODELAY analysis proceeds through two sequential stages, run within CellProfiler and MATLAB (MathWorks), respectively. The ODELAY CellProfiler pipeline imports illumination corrected inverted 8 or 16 bit tif stitched fields of view and proceeds to identify and measure objects within each stitched image over each time course. In the second phase, a MATLAB script registers and tracks objects between time points and then fits object area data the Gompertz function (ƒ),

ƒ=a+be^{-e}^{^{−ct+d}}, (1)

from which growth parameters are extracted by derivative analysis.

The CellProfiler and MATLAB pipelines employed in this study are available as downloadable files, as are a sample input data set and intermediate files from various stages of the analysis (see Supplementary Files online at aitchison.systemsbiology.net/odelay). Using these files, the entire analysis pipeline or specific sections can be explored (see Automated ODDELAY_README.txt). Note that many of the limits employed in the current automated ODELAY analysis pipeline, for example the minimum and maximum diameter cut-offs for detected objects and the proximity threshold applied to ensure robust object grouping through time, are dependent on the experimental set-up (i.e., objective magnification, camera pixel density) and may require optimization when applied to different data sets.

CellProfiler ODELAY Pipeline

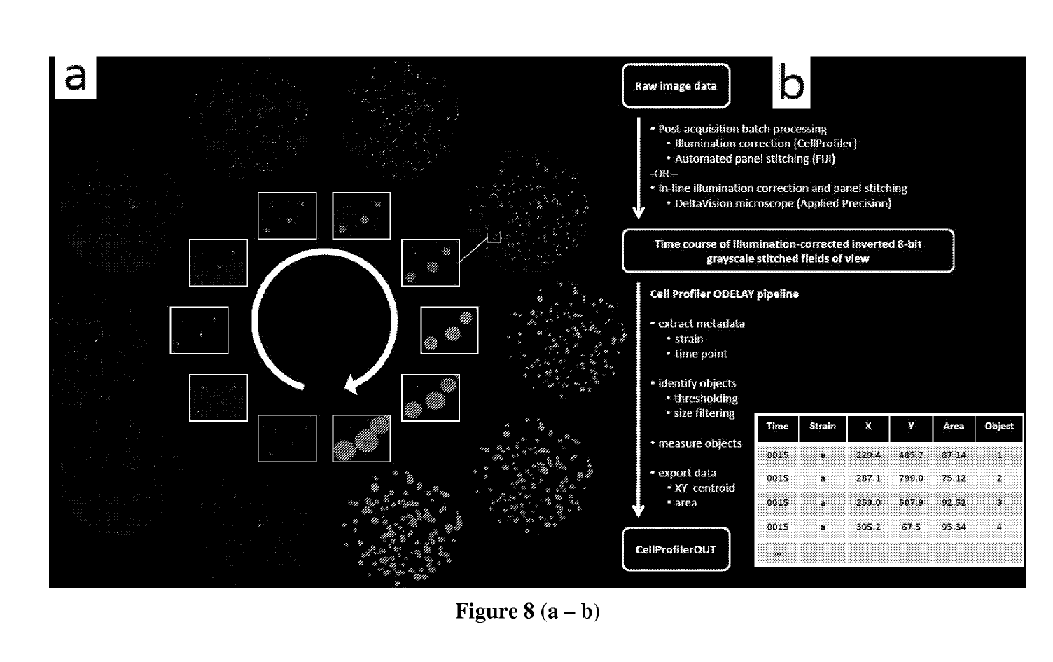

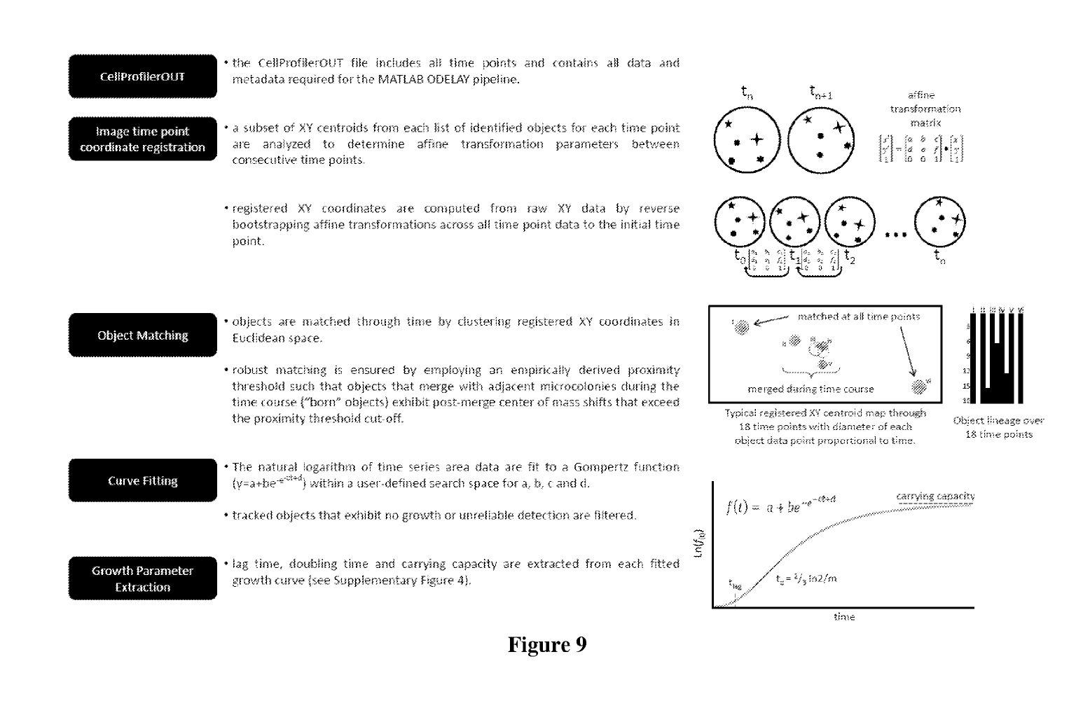

The current CellProfiler ODELAY pipeline, outlined in FIG. 8, analyzes a collection of input images having a Stitched_strain_time.tif nomenclature within the specified default input folder. Input images should be illumination corrected and inverted prior to running the CellProfiler ODELAY pipeline. Alternatively, the pipeline can be modified to perform these additional steps prior to object detection. Objects within each input image are identified using global background thresholding. Robust edge detection is achieved by optimization of the threshold correction factor. Optically dirty images (aberrations in agarose, dust on surface of media etc.) may require optimization of the object diameter cut-off. For the downloadable sample data set, a 1.2 threshold correction factor and 6-1000 pixel object diameter gate were employed. Identified object outlines are overlaid onto input images and saved, allowing qualitative assessment of edge detection fidelity. All object measurements across all time points are exported within a single file for each strain. A summary file of objects detected within each time point image is also saved. Both files are saved within strain specific sub-folders of the default output folder. In preparation for the ODELAY MATLAB pipeline, pertinent data are exported from ODELAY CellProfiler object measurement output files as tab-delimited text files using a CellProfilerOUT-strain.txt naming scheme. Strict adherence to the data array structure detailed in Automated ODELAY_README.txt is required when using the current ODELAY MATLAB pipeline.

MATLAB ODELAY Pipeline

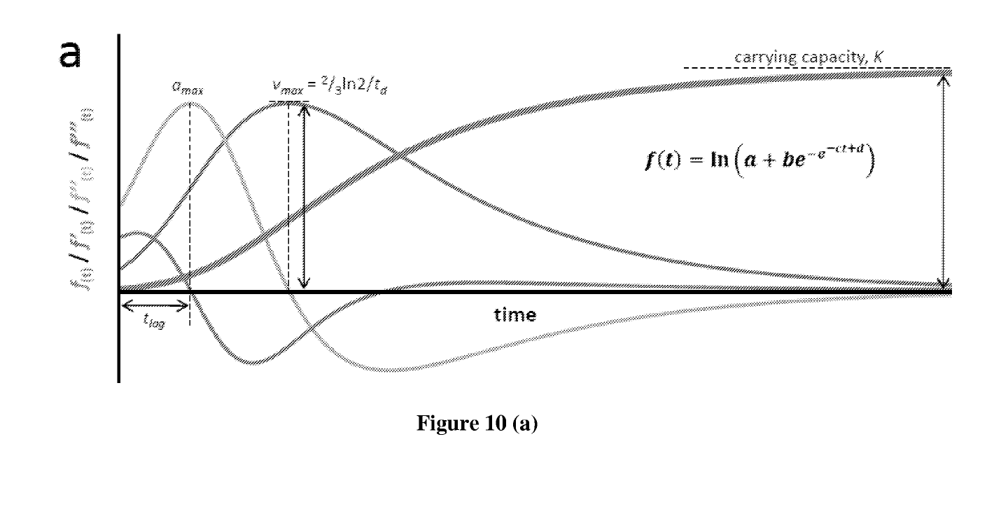

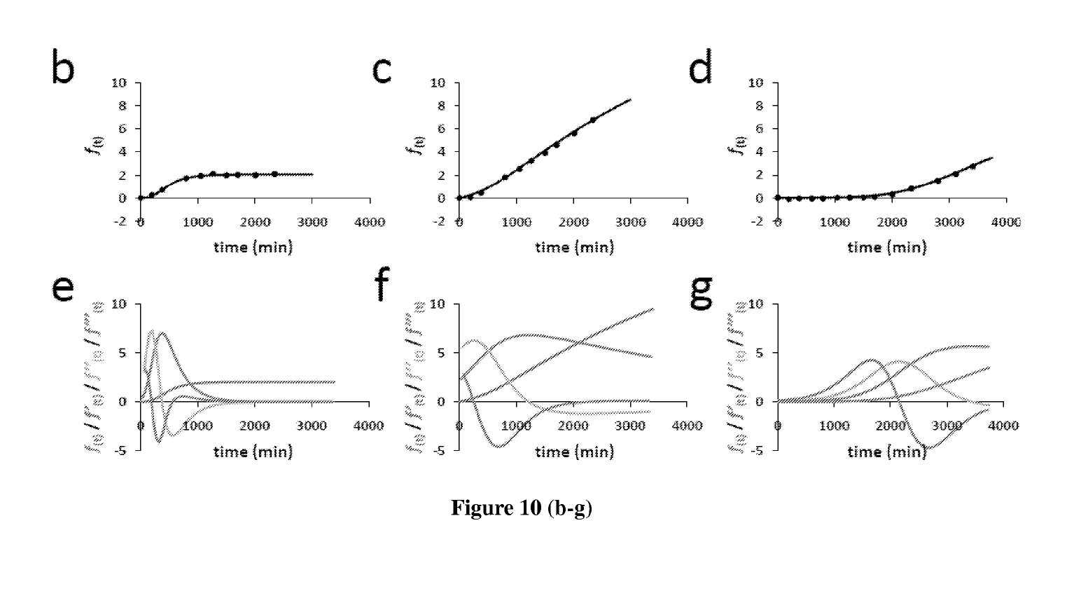

The current CellProfiler MATLAB pipeline is summarized in FIGS. 9 and 10. The only information required for the MATLAB ODELAY pipeline are the position and area of each identified object for each strain at each time point, contained within the CellProfilerOUT.txt file. In the first phase, the positional information for object at each time point are registered due to slight rotation and/or shrinkage of the growth substrate during the time course and variations in stitched image dimensions. Starmatch, a previously described algorithm designed for registration of astronomical images (Riess, A. A., et al., The Astronomical J. (1998) 116:1009-1038) is currently utilized. Starmatch calculates the affine transformation matrices between each time point, using a sub-set of objects XY centroids from each list (currently set to 200 objects per time point). The calculated affine transformations are then applied in a reverse, step-wise fashion to register all positional information back to the initial time point. The fidelity of registration is data dependent using this method because objet XY centroid lists are populated using the smallest objects. That is, the original algorithm was designed to populate lists with the brightest stars in the sky, which have the smallest magnitude values using the historical astronomical metric, and are equivalent to objects with the smallest areas when applied to ODELAY analysis. Due to this inherent data dependence, background noise must be adequately filtered by the CellProfiler pipeline at each time point in order to prevent the appearance or disappearance of a large number of objects between time points from confounding image registration by Starmatch. In the second phase of the ODELAY MATLAB pipeline, registered objects are matched through time by clustering in 2-dimensional Euclidean space (R²) using the groupDataArr MATLAB function (Supplementary Files, ODELAY_MATLAB_pipelie.zip. These are available for download at aitchison.systemsbiology.net/odelay/. A user-definable proximity threshold (θ) is employed in the clustering to ensure that new objects that appear during the time course as the result of the merging of adjacent microcolonies are unlikely to be matched to parent objects (by default θ=10). In the third phase, the Gompertz function (1) is fit to the natural logarithm of area data of objects clustered through time using the gompertzFit MATLAB function (Supplementary Files, ODELAY_MATLAB_pipeline.zip. These are also available for download at aitchison.systemsbiology.net/odelay/. The gompertzFit routine calculates an initial estimate of the Gompertz Function (1) using a coarse grid optimization and then attempts to find a constrained minimum of the function (1) at this initial estimate using the fmincon MATLAB function. In order to proceed to curve fitting objects must be matched at 5 or more time points through the monitored time course. In addition, objects that do not exhibit growth are eliminated from curve fitting. This is achieved by only fitting data for which the maximum observed cross-sectional area of each tracked object is at least two-fold greater than the object's measured cross-sectional area at the first time-point. In the final phase of the ODELAY MATLAB analysis pipeline, the a, b, c and d parameters of the Gompertz function (1) that define each ODELAY growth curve are used to extract microcolony specific growth parameters by derivative analysis (see also FIG. 10). Output data are saved as tab-delimited text files using an ODELAY_strain.txt naming strategy. Column headers in ODELAY_strain.txt output files are annotated in run ODELAY.m and detailed in Automated ODELAY_README.txt. Doubling time (t_{d}) is calculated as follows:

$${t}_{d} = \frac{2}{3} \frac{ln 2}{{v}_{max}} \qquad ( 2 )$$

where νₘₐₓ is the point at which the growth rate, ƒ′(t), reaches the maximum (achieved at ƒ″(t)=0). Lag time (t_{lag}) is defined as the time to reach maximum growth acceleration, aₘₐₓ, where ƒ″(t) is greatest (achieved at the lower value of the two solutions to ƒ″(t)=0). The carrying capacity (K), in pixel area, represents the cross-sectional area of the base of the modeled microcolony projected to stationary phase (ƒ(t) as t→∞) and is calculated as follows:

K=a+b, (3)

Using the calculated a and b parameters of the Gompertz function (1). Microcolony terminal replicative capacity (Z), expressed as generations, is calculated from carrying capacity as:

$$Z = \frac{3}{2} {log}_{2} ( \frac{K}{a} ) \qquad ( 4 )$$

Note that the conversion factors (2/3 for t_{d} and 3/2 for Z) are required because the Gompertz function (1) is fit to microcolony areas, rather than hemispheric volumes. For the automated ODELAY results presented in FIG. 11, post-processing analysis was performed in Excel™ (Microsoft) after elimination of objects that (i) failed detection at either of the first two time points or (ii) yielded quality of fit values (ƒᵥₐₗ) for Gompertz-approximated growth curves of greater than 0.25 . . . ƒᵥₐₗ is calculated as follows

$${f}_{val} = {\sum }_{i = 1}^{N} {log}_{2} ( \frac{{f}_{i}^{e}}{{f}_{i}^{t}} ) , \qquad ( 5 )$$

where ƒᵢ^{e} and ƒᵢ^{t} are the measured and Gompertz-approximated growth curve values (where i ranges over N experimentally measured data points), respectively.

Results

Calculation of Microcolony Volume

FIG. 1(a) depicts the four stages of solid-phase growth parameter derivation and FIG. 1(b) depicts the hemispheric modeling of microcolony expansion. For the representative growth curve shown, the exponential growth phase, or log phase, of a population, N, is defined by the linear region of a plot of log(N) versus time, t. During this phase, population growth follows Equation 1, where t_{d} is the doubling time of the population, Nₜ is the population at time, t, and N₀ is the initial population at entry into exponential phase. Rearrangement of Equation 1 yields Equation 2, which fits the equation of a straight line (y=mx+b); thus, t_{d} is the inverse of the slope (m) of a plot of log₂(N) versus time. The number of cells in the population is proportional to the total volume of cells and replacing population, N, with volume, V, in Equation 2 yields Equation 3. The volume of a yeast microcolony can be modeled as a hemisphere, for which the volume, Vₕₑₘᵢₛₚₕₑᵣₑ, is determined by Equation 4.

Measurements of microcolony cross-sectional area, A, at the surface of growth medium are used to calculate the modeled colony radius, r, as shown in Equation 5, and solving this equation for r and inserting it into Equation 4 yields Equation 6. Equation 7 is derived by inserting the hemisphere volume equation (Equation 6) into Equation 3. Like Equation 3, Equation 7 fits the equation of a straight line for which population doubling time is the inverse of the slope of the best fit line for a plot of

$${log}_{2} ( \frac{2}{3 \sqrt{π}} {A}^{3 / 2} )$$

versus time. Note that the slope of a plot of log₂ (A) versus time is related to the slope of a plot of

$${log}_{2} ( \frac{2}{3 \sqrt{π}} {A}^{3 / 2} )$$

versus time by a conversion factor of 3/2.

Relationship Between Yeast Microcolony Cross-Sectional Area and Doubling Time

ODELAY solid-state growth rate analysis was initially validated against the established method of OD₆₀₀ measurements. Similar to results observed for other proxies of cell population such, a log-plot of microcolony cross-sectional area versus time is linear during exponential growth. It was noted, however, that doubling times obtained from these plots were significantly longer than the doubling times calculated from OD₆₀₀ measurements in liquid culture. Microdissection of a microcolony revealed that the number of cells contained within was significantly greater than the number predicted by cross-sectional area. It was observed that the linearity of log-plots of microcolony cross-sectional area versus time continues long after microcolonies clearly exhibit three-dimensionality. This led to modeling the microcolonies to hemispheres (FIG. 1(b)). In short, rather than plotting the log of microcolony cross-sectional area over time, the log of microcolony volume over time is plotted, where the volume is calculated from the cross-sectional area of microcolonies modeled to the base of a hemisphere (see Equation 7 in FIG. 1). Using this approach, it was found that the doubling times obtained from microcolonies growing on solid media closely match those obtained using established liquid media methods, such as cell counting or OD₆₀₀ measurements.

Quantitative Growth Rate Analysis of Yeast Microcolonies—Establishing ODELAY Proof of Principle

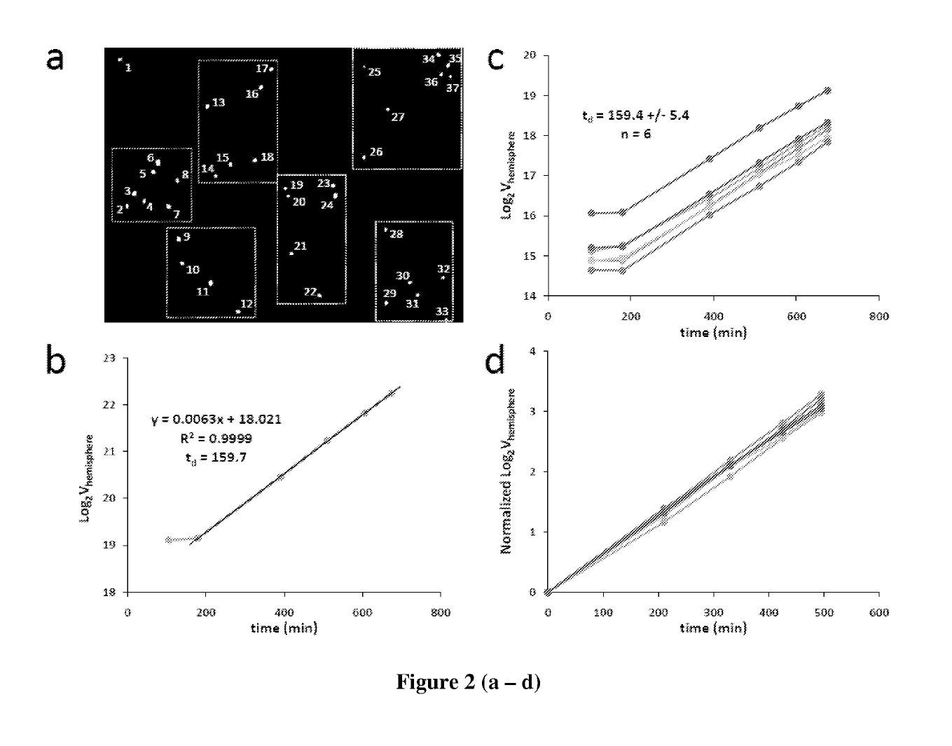

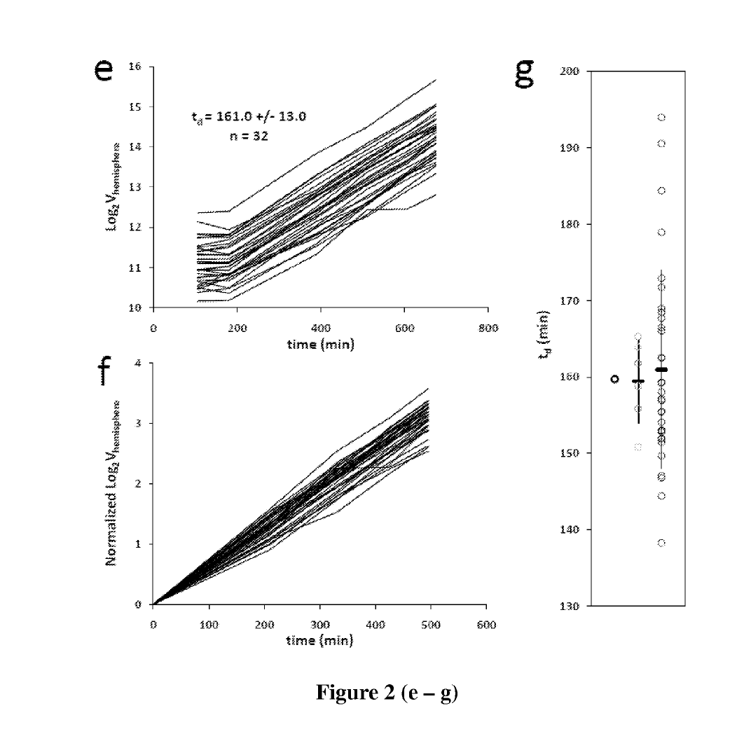

Several pilot experiments were conducted to evaluate the precision, accuracy and dynamic range of solid phase cell doubling analysis by microcolony cross-sectional area measurements. In the first, a single field of view (FOV) was imaged over a 12 hour time course and assessed the growth rates of individual microcolonies, small groups of microcolonies and all microcolonies in the FOV. The results, shown in FIG. 2, demonstrate the strong linear correlation between log-plots of hemisphere-modeled microcolony volume and time. FIG. 2(a) shows annotated field of view (FOV) containing 37 individual yeast microcolonies, each seeded from 1-4 cells of the yeast strain M₃16/1A (Amberg, D. C., et al., EMBO. J. (1993) 12:233-241). FIG. 2(b) shows that after a brief lag period, the plot of the log₂ of calculated microcolony hemispheric volume for the entire FOV versus time attains linearity and, from this linear region, the population doubling time of 159.7 minutes is extracted. FIG. 2(c) shows similar analyses performed on smaller regions of the total FOV (indicated by colored boxes in (a)), each containing 4-7 microcolonies. From these six sub-regions, a doubling time of 159.4+/−5.4 minutes was obtained. FIG. 2(d) shows the linear regions of plots shown in (c) were normalized at the origin. FIG. 2(e) shows individual analysis of 32 microcolonies from the initial field of view yields a doubling time of 161.0+/−13.0. Colonies 5, 6, 23 and 24 were eliminated from the analysis due to convergence at late time points, and colony 25 was removed due to lack of any measurable growth. FIG. 2(f) shows the linear regions of plots shown in (e) were normalized to the origin. FIG. 2(g) (left to right) shows summary of doubling times calculated from the entire FOV, sub-regions of the FOV and individual microcolonies. Individual data points are indicated by open circles, the average doubling time by cross-hatches and the standard deviations, for sub-regions and individual microcolonies, by vertical lines.

Regardless of whether microcolonies were analyzed individually, in small groups or as the entire FOV, a doubling time of ˜160 minutes was obtained. These data indicate that microcolony doubling times are unaffected by variation in the number of seeded cells from which microcolonies form. The precision of doubling times extracted from these plots is greater when small groups of microcolonies are used (FIG. 2(d)) rather than individual analysis for each microcolony (FIG. 2(f)), but it remains undetermined whether the increase in variance associated with individual microcolony analysis is due to biological or experimental noise.

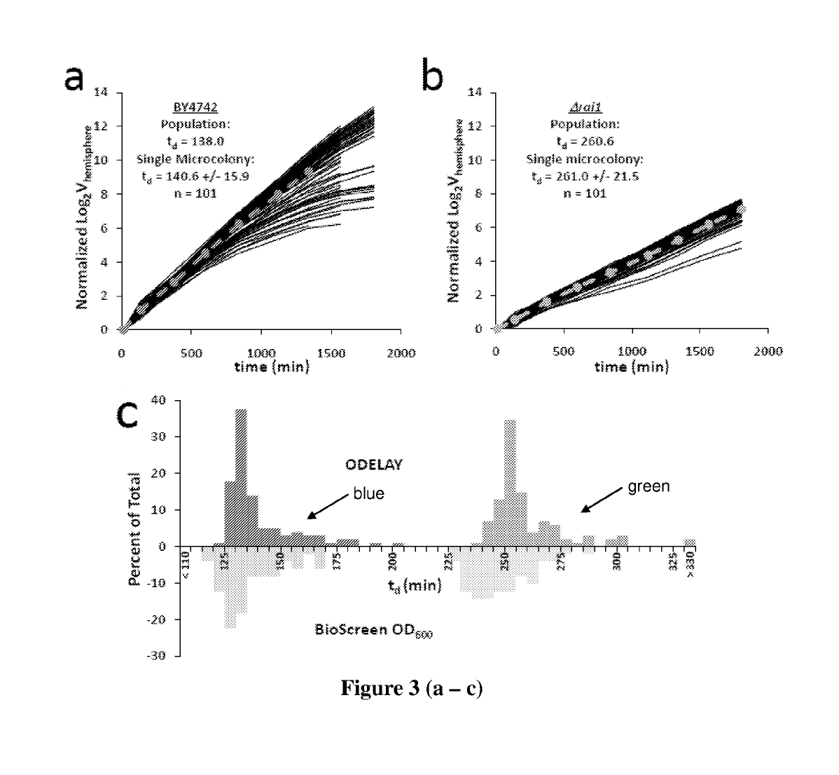

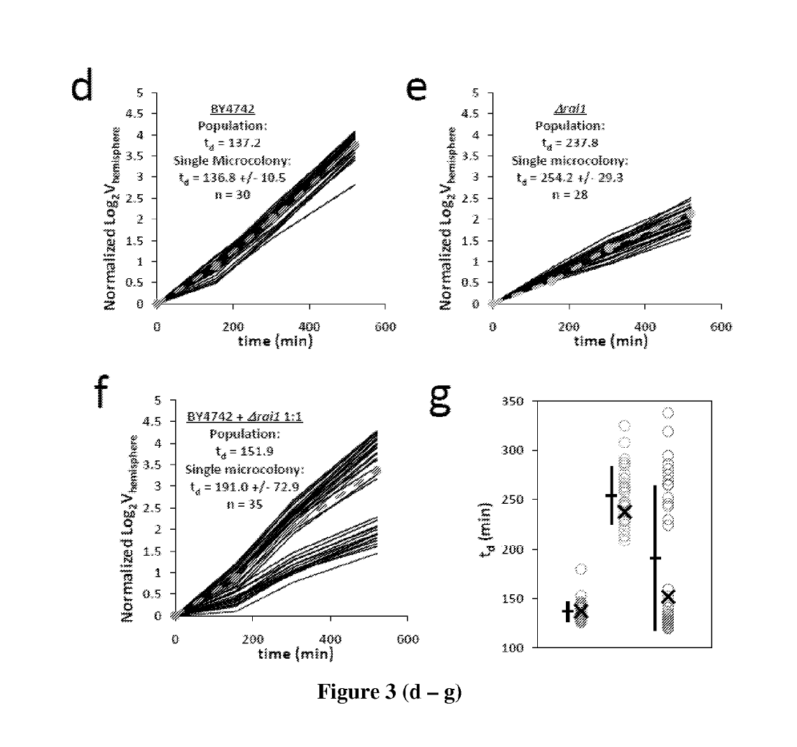

Doubling times calculated by ODELAY were directly compared to liquid culture OD₆₀₀ measurements for the wild-type yeast strain, BY4742, and a slower growing null mutant strain, Arai 1. For each strain, 49 liquid culture doubling times and 101 ODELAY solid-phase doubling times were calculated (FIG. 3). In FIG. 3(a) shows BY4742 or (b) Δrail ODELAY 30 hour growth curves for individual microcolonies (black lines) and the population as whole (colored dashed line) with calculated doubling times inset. Data points for the population curves indicate sampled time points. FIG. 3(c) shows distribution analysis of ODELAY microcolony doubling times (n=101) and BioScreen™ OD₆₀₀ doubling times (n=49) for BY4742 cells (blue) and Δrail cells (green) using 5 minute bin intervals and frequency values normalized as fraction of total. Percent of total for BioScreen™ data are plotted as negative values for display purposes. FIGS. 3(d), (e) and (f) show ODELAY analyses of individual microcolonies (black lines) and the population as a whole (colored dashed lines) for BY4742 (n=30), Δrail (n=28) and a 1:1 mix of BY4742 and Δrail (n=35), respectively. Data points for the population curves indicate sampled time points. Individual microcolonies in the heterogeneous sample stratify into two populations with distinct growth rates that correlate with the observed growth rates of BY4742 or Δrail. FIG. 3(g) shows summary of the distribution of doubling times obtained from individual microcolony ODELAY analyses of BY4742 cells (blue), Δrail cells (green) or the 1:1 mix (red). The mean and standard deviation for each distribution is plotted to the left and the FOV (population) doubling time is indicated by an X. The heterogeneity of the 1:1 mix population is masked by full FOV (population) analysis but is readily evident when individual microcolonies are analyzed.

For ODELAY, these measurements were obtained from two spots, each about 3 mm dia., pinned using a 384 sample density pinning device. For OD₆₀₀, the measurements were made using two BioScreen™ honeycomb plates, each approximately the size of a standard 96-well microtiter plate. The values and distributions of the doubling times calculated by ODELAY and OD₆₀₀ were comparable for both the fast and slow growing strains (FIG. 3(c)). The most notable exception is that ODELAY identified slow growing outliers because ODELAY microcolony growth curves are derived from single cells unlike liquid culture OD₆₀₀ curves, which are representative of a seed population of cells.

Using the same data, the dynamic range of ODELAY was assessed. Implicitly, the lower limit of the assay is a single cell as the spotting process produces microcolonies seeded from 1-4 cells, which are readily detected at the employed magnification. The outer limit of ODELAY was gauged by identifying the point at which log-plots of hemisphere-modeled microcolony volume lose linearity over the 30 hour time course (FIG. 3(a)). No clear entry into stationary phase was observed for volumetric microcolony analysis for the entire FOV (population) throughout this time course, during which time cells underwent great than 10 doublings. Most microcolonies maintained a constant rate of exponential growth throughout the 30 hour growth period; however, the expansion of a subset of microcolonies waned over time and the prevalence of waning increased over time. The loss of linearity observed for individual microcolonies at later time points is not related to the convergence of adjacent microcolonies, the location of microcolonies within the spot, nor is it an image processing artifact. Another possibility is that there may exist an upper limit to colony size outside of which hemispheric modeling cannot be applied. This scenario is unlikely given that the majority of individual microcolonies exhibited exponential growth throughout the monitored time frame. Another more favorable interpretation is that the loss of linearity represents growth deceleration at early stationary phase. This is unlikely to account for all cases or growth arrest given that arrest has been observed as early as 4 doublings, which is far below the nutrient-limited carrying capacity. Regardless, the observation of growth deceleration highlights the sensitivity of ODELAY, relative to liquid culture analysis. It is unlikely that this phenotype would be detectable at the level of overall population growth by liquid culture or biofilm analysis because it occurs in only a small proportion of cells and renders them competitively unfit.

Together, these data suggest that the limiting factor at the upper end of the dynamic range of ODELAY is microcolony convergence, which is dependent on the initial seed density, rather than the carrying capacity of the solid growth medium. Due to differences in strain doubling times, the dynamic range is best defined by the total number of doublings required to reach the upper limit starting from a single cell. At optimal seed density (˜50-100 cells per mm²), the dynamic range of ODELAY is 8-12 doublings—from a single cell up to 250 or as many as 4000 cells, which compares favorably to the 3-5 doubling dynamic range attainable by most currently available technologies.

Information specific to individual microcolonies yields parameters describing population heterogeneity, making ODELAY a powerful approach to the study of fundamental biological phenomena such as chronological aging, epigenetic switching, replicative capacity, response to stress and other perturbations that alter population variability and/or viability. To demonstrate the utility of ODELAY in assessing population heterogeneity, the growth parameters of wild type BY4742 and Δrail were compared but also included a 1:1 mixture of these two strains (FIG. 3(d)-(f). By FOV analysis it is unclear that the 1:1 mix is a heterogeneous population (FIG. 3(g)), similar to what is observed by OD₆₀₀ growth rate analyses of heterogeneous cultures. Since the growth of the subpopulation of Δrail microcolonies is negligible relative to the growth of the wild-type cells, the 1:1 mix sample exhibits a doubling time close to the observed doubling time of wild-type microcolonies. It is only when individual microcolonies are analyzed that it becomes apparent that the 1:1 mix contained two sub-populations of cells at approximately equal relative abundance in the seed population (FIG. 3(f)).

Derivation of Lag Time by ODELAY

Another key growth parameter that can be determined using ODELAY is lag time, the period of adaptation or conditioning prior to exponential growth. The length of lag is variable in different genetic and environmental contexts and changes in lag time can arise due to reduced overall strain fitness or from an increased or decreased ability to respond to specific environmental cues. FIG. 4(a) shows idealized log₂ plot of hemispheric volume versus time (red) with extrapolation of the linear region, the period of exponential growth, to the y-intercept (black). The lag time is indicated (t_{lag}) and is calculated from three parameters: the slope (m) and y-intercept (b) of the best fit line describing the linear region and the log₂ value of the initial microcolony hemispheric volume (log₂V₀). FIG. 4(b) shows full FOV (population) ODELAY analyses of a wild-type strain, BY4742, growing on media containing galactose as the sole carbon source. Cells were preconditioned by liquid culture growth in galactose (red) or glucose (blue) media. Black lines represent the best-fit lines of each respective linear region. The calculated doubling times and lag times for galactose or glucose preconditioned cells are shown. FIGS. 4(c) and (d) show individual microcolony ODELAY analysis for galactose (n=40) and glucose (n=42) preconditioned cells, respectively. Calculated doubling times and lag times (+/−standard deviation) are shown. FIG. 4(e) shows plots of doubling time (t_{d}) versus lag time (t_{lag}) for microcolonies from cells preconditioned in galactose (red) or glucose (blue). FIG. 4(f) shows stitched image of spots containing ˜200 microcolonies growing on galactose media 32 hours subsequent to their initial spotting from glucose or galactose liquid cultures. The region analyzed by ODELAY is bounded by the dashed white line.

Derivation of lag time by ODELAY, detailed in FIG. 4(a), requires no additional data from those used to calculate doubling times, with the exception that lag time determination necessitates the acquisition of an early time point. Ideally, this early time point is acquired at time zero but, for practical purposes, any time prior to the initial onset of cell division is suitable (i.e., prior to the time at which the majority of singly spotted cells are observed to proceed through their first cell division). Through ODELAY analysis, outliers with highly variable, expanded or contracted lag periods can be identified by assessing the distribution of lag times for microcolonies of a given strain (lag time variability) as well as relative lag between tested strains.

To demonstrate the quantification of lag time by ODELAY, we exploited the well-studied and highly regulated response of yeast to a carbon source shift from the preferred source, glucose, to an alternative source, galactose. This shift is characterized by a cessation of growth, during which time the normally repressed galactose utilization machinery, including the galactose transporter, is produced. Exponentially growing yeast preconditioned in either glucose or galactose liquid medium were spotted onto solid media containing galactose and analyzed by ODELAY (FIG. 4). Cells preconditioned in galactose media exhibited a highly synchronized response characterized by short lag times. In contrast, more pronounced and variable lag times were observed for cells that were not primed for growth in galactose. Despite this large preconditioning-dependent difference in lag times, doubling times were relatively unaffected. Once glucose grown cells acclimated to the shift to galactose and entered exponential phase, they doubled at rates similar to those observed for galactose preconditioned cells but with greater variance.

Multiparameter Application of ODELAY

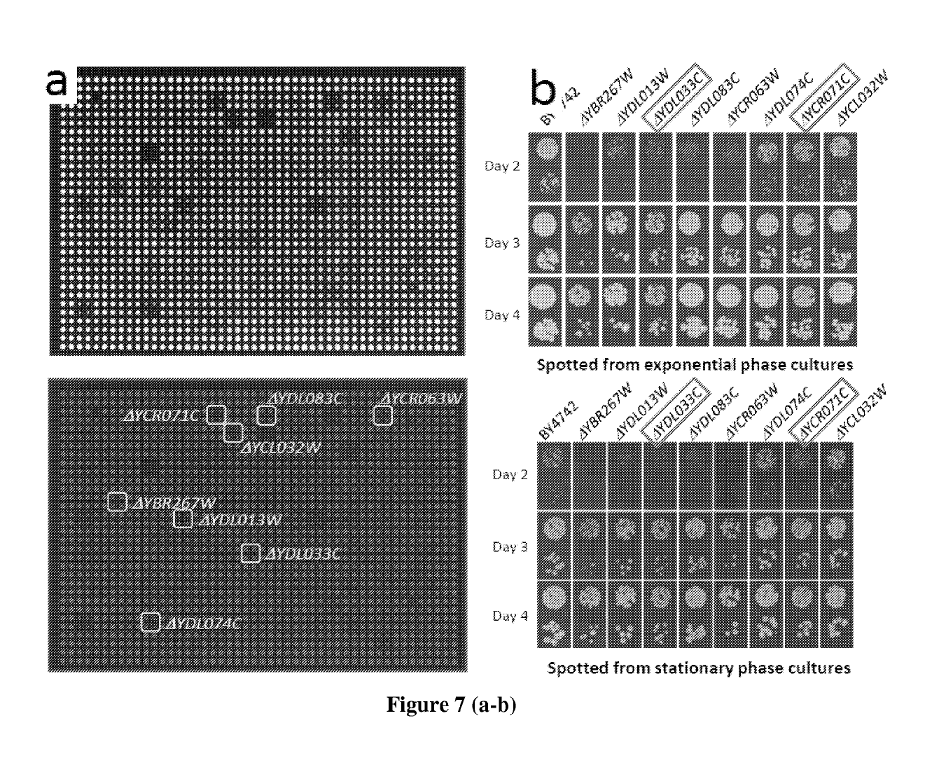

The ability of ODELAY to extract doubling times and lag times for populations of cells growing on solid media and determine population heterogeneity through the analysis of individual microcolonies was demonstrated. To demonstrate multiparameter growth rate analysis by ODELAY, BY4742 to a small subset of strains from the systematic yeast knock-out library were compared. In total, eight strains were selected from a single plate containing 384 knock-out strains. Strain selection was based solely on qualitatively retarded growth and the only other information known about these strains a priori was that each contained a stable, unique gene knock-out in the BY4741 background. The relative fitness for some strains was ambiguous when analyzed by cell spotting assays. FIG. 7 depicts slow-growing strains selected for multiparameter ODELAY analysis. FIG. 7(a) shows 1536 density biofilm array containing 384 yeast null mutants pinned in quadruplicate from which 8 qualitatively slow-growing strains were selected for ODELAY analysis. Shown are the raw image (top) and an adjusted image overlaid with the locations and identifications of the 8 selected strains (bottom). FIG. 7(b) shows serial dilution cell spotting results of selected slow-growing strains. For two null mutants (bounded by red boxes), cells spotted from exponentially growing seed cultures (top) or seed cultures allowed to reach stationary phase (bottom) yielded different apparent relative growth rates. The doubling time, lag time and viability of each strain after moderate chronological aging were quantitatively determined by ODELAY.

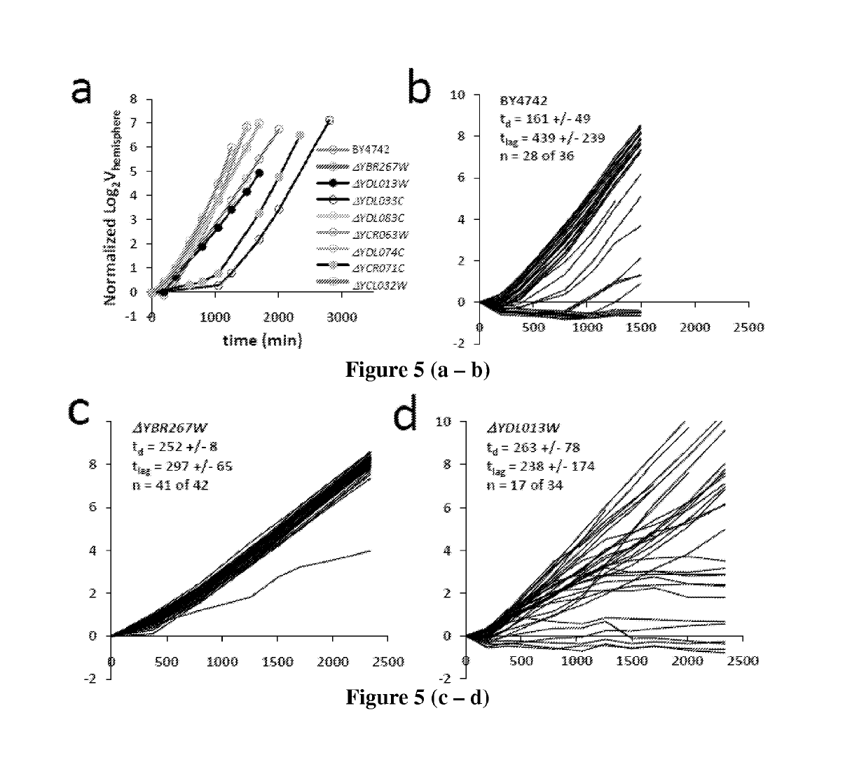

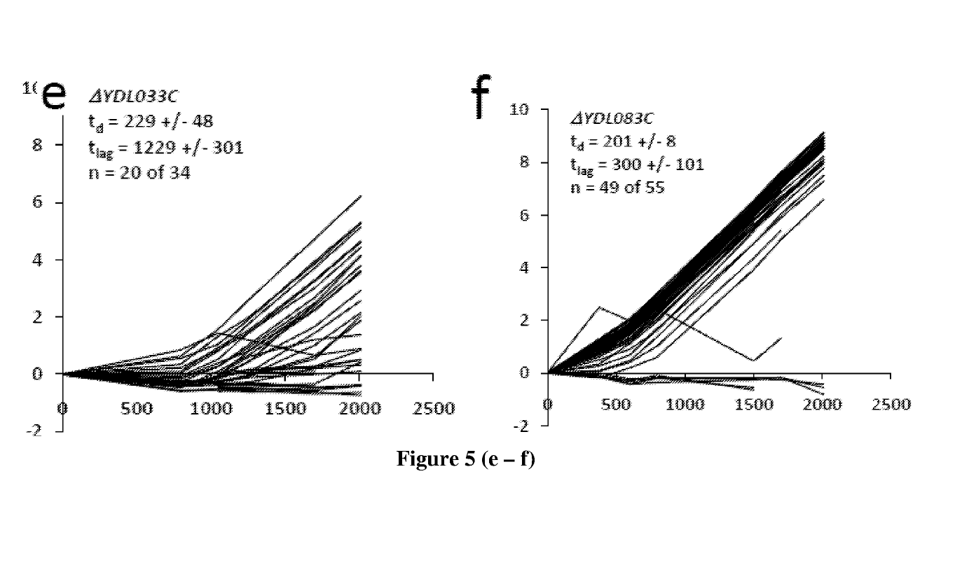

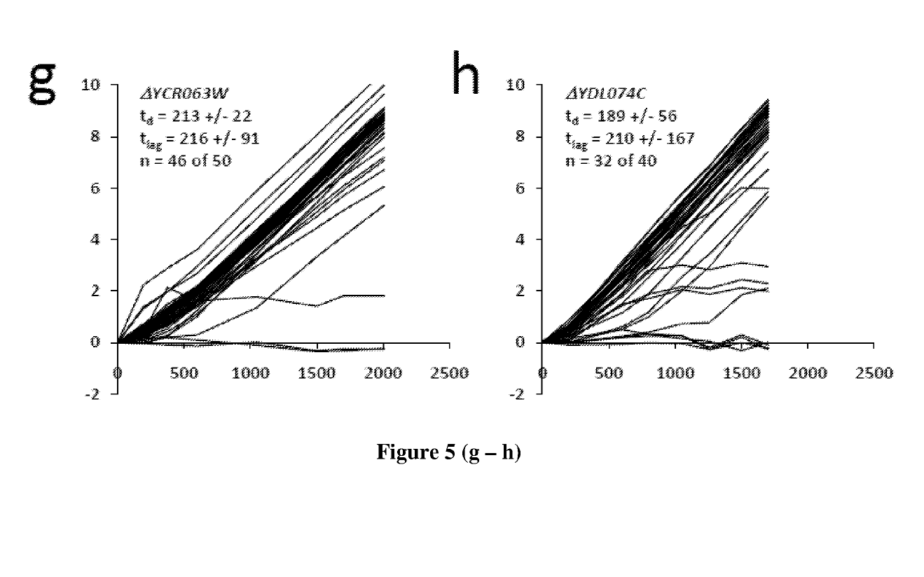

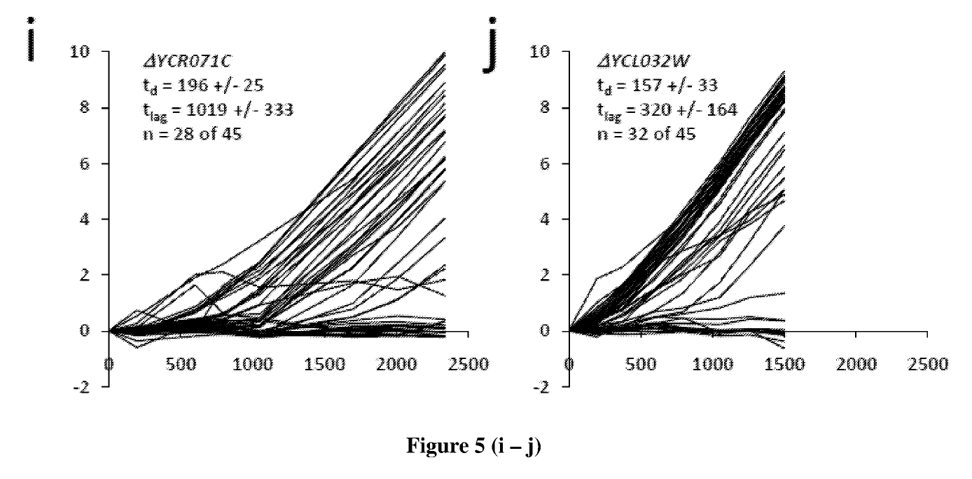

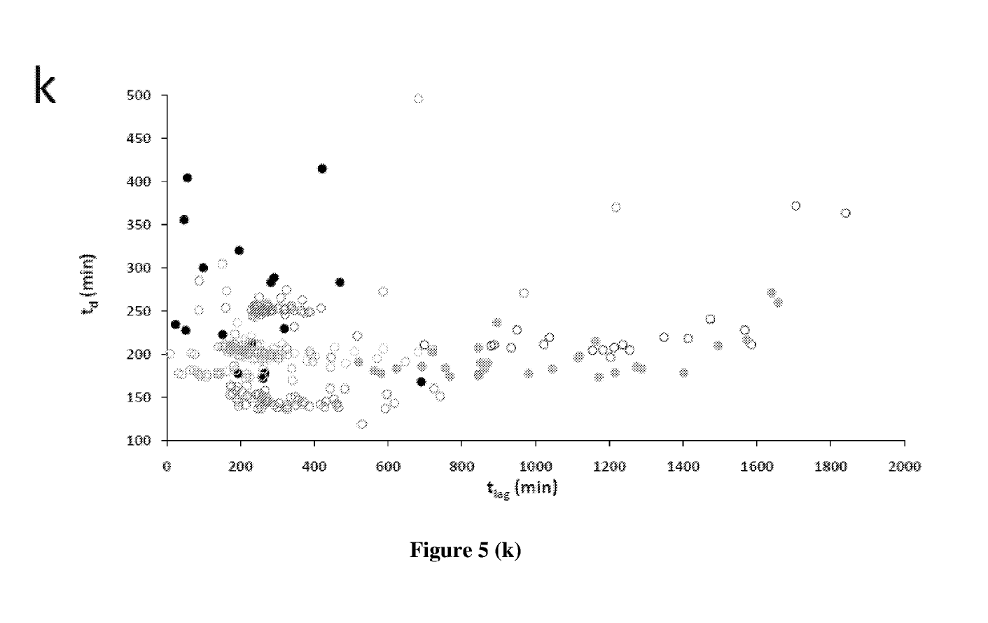

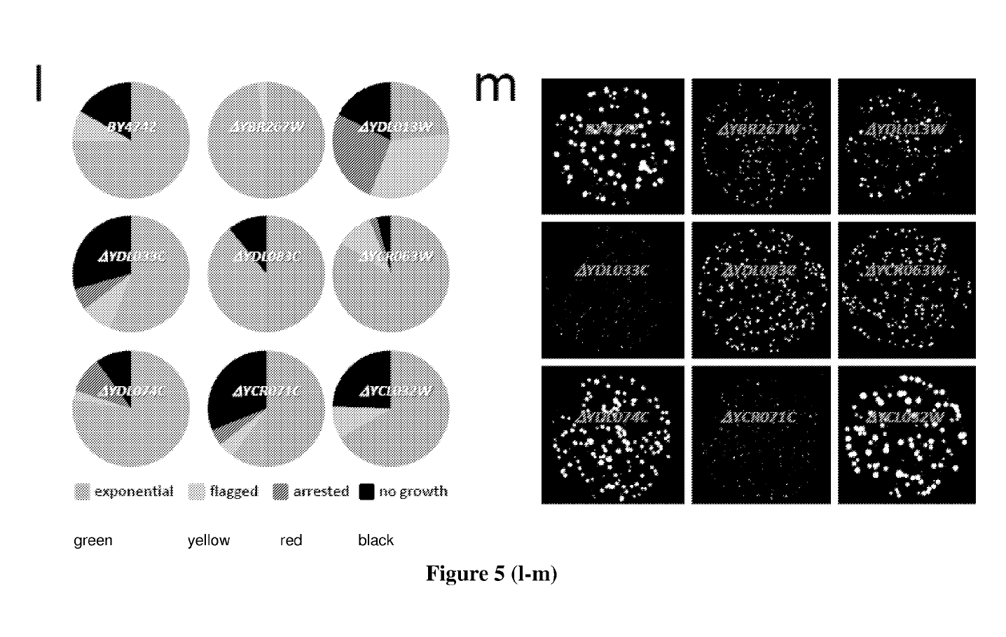

This analysis, summarized in FIG. 5 with specific examples highlighted in FIG. 6, underscores how complex relationships between differences in doubling time, lag time, replicative capacity and/or viability can underlie ambiguities in relative fitness assessment by non-dynamical methods and, when assessing gene-gene and gene-environment interactions, provide details of the nature of interactions. FIG. 5(a) shows FOV (population) ODELAY growth curves for wild-type yeast and 8 qualitatively slow-growing null mutant strains. FIGS. 5(b)-(j) show individual microcolony ODELAY growth curves for the 9 strains tested. The mean and standard deviation of the lag times and doubling times, as well as the number of microcolonies used for these calculations relative to the total number of microcolonies in the FOV for each strain is inset. FIG. 5(k) shows scatter plots of t_{d} versus t_{lag} for each strain. Only data points used for mean and standard deviation calculations are shown. Legend as shown in FIG. 5(a). FIG. 5(l) shows qualitative analysis of microcolony fate. Microcolonies were scored as exponential (green) if, once attained, exponential growth continued throughout the monitored time course, flagged (yellow) if growth waned but did not stop, arrested (red) if growth appeared to stop completely at any point after exponential growth was attained, or no growth (black) if microcolonies exhibited no apparent growth over the entire time course. FIG. 5(m) shows stitched images of spots, each containing 100-200 microcolonies, at the 28 hour time point.

FIG. 6(a) shows top row Binarized image time course demonstrating a ΔYCR071C cell (pseudo-colored red throughout) entering exponential growth after ˜2000 minutes of dormancy. bottom row Unbinarized data for the region bounded by a white box in the left-most panel is magnified. FIG. 6(b) shows extension of the ODELAY curve of the severely lagged ΔYCR071C microcolony to 4000 minutes overlaid over the ΔYCR071C growth curves presented in FIG. 5(i). FIG. 6(c) shows image time course for a representative FOV of ΔYDL013W microcolonies. Arrested microcolonies are indicated by arrows at the 2010 minute time point. FIG. 6(d) shows overlaid ODELAY growth curves and (e) doubling time versus lag time distributions for wild-type (blue), ΔYBR267W (red), ΔYDL033C (black) and ΔYBR267WΔYDL033C (green) cells after seven days of chronological aging. Of the 50 microcolonies monitored for each strain, the number that exhibited growth during the time course are indicated in the legend and only these are displayed.

For example, due to protracted lag times, the ΔYDL033C and ΔYCR071C strains appear to grow far more slowly than the other strains at early time points; nonetheless, the log phase doubling times of ΔYDL033C and ΔYCR071C microcolonies are not dramatically slower than most of the other qualitatively slow-growing strains tested. For non-dynamical cell spotting or biofilm analyses, this extended lag manifests as an apparent growth defect only at early time points using seed cultures that have been allowed to reach stationary phase prior to spotting. An extreme example in which a ΔYCR071C cell lay dormant for over 30 hours prior to entry into exponential growth is shown in FIG. 6(a). The microcolony formed by this cell continues to grow exponentially for 12 doublings at a rate equal to that observed for its less severely lagged isogenic neighbors (FIG. 6(b)). That the exponential growth of this microcolony continues even after it is surrounded by converging and much larger adjacent microcolonies underscores the enviable dynamic range of ODELAY with respect to the carrying capacity of the local environment and the minimal potential for edge effect type artifacts when using this method of growth assessment.

Additional phenotypes were revealed in this analysis. Among them is the high rate of growth cessation observed for ΔYDL013W microcolonies (FIG. 5(d)) relative to wild-type cells (FIG. 5(b)) that resulted in a broad distribution of ODELAY-derived doubling times for this mutant strain. ODELAY growth curves for arrested microcolonies mimicked entry into stationary phase despite being well below the nutrient limiting carrying capacity, suggestive of a replicative capacity defect in this mutant (see also FIG. 11(b)). In several cases, seeded cells were observed to undergo as few as 4 doublings (from a single cell to ˜16 cells) and then stop growing for the remainder of the time course. This growth cessation, observed only in a subpopulation of microcolonies, is suggestive of phenotype lag resulting from a catastrophic event in a progenitor cell(s) from which the microcolony developed. In support, microcolonies derived from ΔYDL013W cells were less often uniformly round, producing ellipses or crescents, supportive of poor viability of this null mutant during logarithmic growth (FIG. 6(c)). Also of note was the tight correlation of doubling times and lag times observed in the ΔYBR267W strain (FIG. 5(c)). Relative to the BY4742 control, seeded cells of this mutant retained viability through moderate chronological aging and uniformly entered exponential growth, albeit at markedly reduced rates.

To test for the combinatorial effects of individual mutant phenotypes, a ΔYBR267W strain was mated with a ΔYDL033C strain to generate a heterozygous diploid and, after sporulation of the diploid, we performed ODELAY analysis on four sister spores representing each of the possible meiotic progeny (FIG. 6(d)). This analysis reveals that, whereas the ΔYBR267WΔYDL033C double mutant grew more slowly than either of the single mutant strains, the extended lag phenotype associated with the ΔYDL033C null strain is less penetrant in the double mutant (FIG. 6(e)). Through biofilm fitness assessment, this null mutant combination is reported to be a positive genetic interaction. Given that deletion of YBR267W has been reported to render yeast cold-sensitive, the room-temperature growth results may not be directly comparable to previous reports. Nonetheless, the results support this finding and further suggest that the positive genetic interaction is likely due to lag suppression rather than through alleviation of a growth rate defect.

Automation of ODELAY Analysis

To improve the throughput and robustness of the ODELAY method, an automated pipeline was developed that encompasses acquisition and processing of images, identification and measurement of microcolonies at each time point, matching of microcolonies through time and extrapolation of growth parameters from growth curves. This current platform, described is outlined in FIGS. 8 through 11. It employs theoretical approximation of ODELAY growth curves using the Gompertz function as an unsupervised method to extract growth parameters (19: Zwietering, M. H., Appl. Environ. Microbiol. (1990) 56:1875-1881). All files required for execution of automated ODELAY analysis, as well as a demonstrative data set, are available as Supplementary Files online at aitchison.systemsbiology.net/odelay.

FIG. 8(a) depicts Representative stitched, illumination corrected time course data images for all cells seeded within a single spot (outer ring) and a zoomed region from each time point image (inner ring). The zoomed region is marked by the white rectangle in seventh time point image. The red lines in zoomed images circumscribe objects identified and analyzed by the Cell Profiler ODELAY pipeline. The loss of detection of the non-growing cell in the upper left portion of the zoomed region at later time points was not uncommon for inviable seeded cells and did not limit the ability of the automated pipeline to identify these objects as non-growing. FIG. 8(b) depicts data flow of image processing and object detection pipeline.

FIG. 9 shows object time point registration, matching and growth curve modeling. The MATLAB ODELAY analysis pipeline utilizes information contained within the CellProfilerOUT file to (i) register each image relative to the initial time point, (ii) match objects through time, (iii) use area data of matched objects though time to fit ODELAY growth curves to a Gompertz function and (iv) extract the lag time, doubling time and carrying capacity from each modeled ODELAY growth curve by derivative analysis (summarized in FIG. 10).

FIGS. 10(a)-10(g) show growth parameter extraction from modeled ODELAY growth curves. FIG. 10(a) shows the natural logarithm plot of a representative ODELAY growth curve modeled to a Gompertz function (ƒ(t), blue) versus time and the first derivative (ƒ′(t), red), second derivative (ƒ″(t), green) and third derivative (ƒ′″(t), purple) of the natural logarithm of this function are plotted. Note that the plots have been scaled vertically relative to one another for display purposes. Doubling time, td, is calculated as 2/3 ln²/νₘₐₓ, where νₘₐₓ is the point at which the growth rate, or first derivative, reaches maximum. This maximal growth rate is achieved ƒ″(t)=0. Note that the scaling factor, 2/3, is required because microcolony areas are modeled by the Gompertz function, rather than microcolony hemispheric volumes. Lag time, t_{lag}, is defined as the time to reach maximum growth acceleration, aₘₐₓ, where ƒ″(t) is greatest. Maximal acceleration is obtained at the first of the two x-intercepts of the third derivative (ƒ′″(t)=0). The carrying capacity (K), expressed as pixels, represents the cross-sectional area of the base of the modeled microcolony projected to stationary phase (ƒ(t) as t→∞) and is equal to the sum of the a and b values of the Gompertz function. FIGS. 10(b)-(d) show representative modeled ODELAY growth curves for microcolonies with reduced carrying capacity (left), typical carrying capacity (middle) and extended lag time (right), respectively. The data points to which curves were fit are indicated by black circles. FIGS. 10(e)-(g) show derivative analysis of the ODELAY growth curves shown in FIGS. 10(b)-(d), respectively. As in (a), derivative plots have been scaled vertically for display purposes and, therefore, y-axis values are specific only to the modeled growth curve (ƒ₍ₜ₎).

FIGS. 11(a)-11(d) show comparison to manual analysis. FIG. 11(a) shows doubling time versus lag time distributions (compare to FIG. 5(k)) extracted by automated ODELAY for the nine strains analyzed manually and presented in FIGS. 5 and 6. Data density is approximately four-fold greater for automated analysis because all seeded cells within each spot were analyzed using the automated method, whereas manual analysis was restricted to sub-regions of the total seeded spot. FIG. 11(b) shows quantification of carrying capacity by automated ODELAY analysis can reveal replicative capacity reduction and augmentation. Microcolony terminal replicative capacity, expressed as generations, is calculated as 3/2 log 2[(a+b)/a] from the constants a and b of the modeled Gompertz function, ƒ=a+b^{-e}^{^{ct+d}}. The conversion factor 3/2 is required because microcolony area, rather than hemispheric volume, is fit to the Gompertz function. For each chronologically aged strain, the proportion of all viable seeded cells is plotted versus terminal replicative capacity. The dynamic range of a typical ODELAY experiment (12 doublings) is shaded in blue and, thus, replicative capacities outside of this range are projected. Reduced replicative capacity (early-onset arrest) was observed for AYDL013W cells, as almost half of viable seeded AYDL013W cells produced microcolonies that arrested within the typical dynamic range of the assay, whereas less than 20% of viable wild-type cells (BY4742) reached terminal replicative capacities at less than 12 generations. In contrast, AYBR267W cells appear to be protected from early-onset arrest relative to the other strains tested, as AYBR267W cells were the least likely to arrest during the monitored time course. The replicative capacity phenotypes observed for AYDL013W and AYBR267W cells by automated ODELAY analysis agrees with the semi-quantitative manual assessment of these data (compare to FIG. 5(l)). FIG. 11(c) shows summary of doubling times and lag times obtained for the nine tested strains (compare to FIG. 5(b)-(j). Median analysis was performed using data from all viable cells (Nₜₒₜₐₗ), whereas data sets were trimmed of the top and bottom 5% of values (N_{0.9}) prior to calculation of average+/−standard deviation. FIG. 11(d) shows doubling time versus lag time distributions (average+/−standard deviation) for _{N0.9} data (top and bottom 5% trimmed).

A comparison of the manually acquired growth parameters presented in FIG. 5(k) to those obtained using our automated ODELAY platform (FIG. 11(a)) reveals that the automated method yields results similar to manual analysis. Notably, unlike the manual analysis, in which it was limited to qualitative descriptions of carrying capacity (FIG. 5(l)), the automated method provides quantitative measurements of this growth parameter (FIG. 11(b)). Carrying capacities extracted from modeled ODELAY growth curves are most often reached at times far beyond the monitored time course and, thus, caution should be exercised when examining phenotypes associated with increased carrying capacity. In contrast, reduced carrying capacity is a sensitive indicator of strains with replicative capacity defects because microcolonies are observed to arrest during the monitored time course (FIG. 11(b)).

As shown in this Example, ODELAY was validated by comparison to the well-established OD₆₀₀ liquid culture method. These experiments revealed that, while both techniques yield doubling time distributions of similar precision and accuracy, only ODELAY is capable of resolving growth rates for mixed populations of cells into distinct sub-populations.

As with other methods of growth analysis, there are caveats associated with ODELAY. Extraction of cell doubling time by ODELAY relies on the assumption that microcolonies are hemispheres, that this shape is maintained throughout microcolony development and unaffected by changes in growth condition and/or genetic background. Comparison of doubling times obtained by hemispheric modeling to those obtained by OD₆₀₀ measurement (FIG. 3(c)) suggests that yeast microcolonies take on a hemispheric shape during development. There will certainly be exceptions in yeast, or other colony forming microorganisms; however, similar to limitations in OD₆₀₀ analysis when applied to flocculent mutant strains, such exceptions may, in fact, yield informative phenotypic information. Furthermore, microcolony area data can be analyzed by ODELAY without three-dimensional extrapolation, in which case the ability to derive absolute doubling time is sacrificed but quantitative growth parameters describing microcolony cross-sectional area expansion are nonetheless obtained. Indeed, in light of the potential for run to run variability, high throughput application of this method will likely employ relative rather than absolute assessment of growth.

There are several practical benefits to high-throughput growth rate analysis on solid media by the method described herein as an alternative to liquid media, including increased sample density and reduced handling and materials. The major benefit of ODELAY, however, is the ability to monitor, for up to hundreds of thousands of individual microcolonies, the growth parameters that together define their expansion from single seeded cells, allowing a resolution beyond that permitted by non-dynamical metrics of fitness.

In this application, “a”, “an”, and the like are intended to mean one or more than one unless it is clear from the context that some other meaning is intended. All documents cited herein are hereby incorporated herein by reference.

The following materials available online may be helpful in understanding the invention. They may be found on the web at aitchison.systemsbiology.net/odelay/.
AutomatedODELAY_README.txt
ODELAY_SampleDataSet.zip (800 Mb)
ODELAY CellProfiler pipeline input data files. This data set is comprised of the 102 stitched, inverted and illumination corrected 8-bit tif image files used in the analysis presented in FIGS. 11(a)-11(d).
ODELAY_CellProfiler_pipeline.cp (8 kb)
This current version of the ODELAY CellProfiler pipeline is described in Methods online. Refer to AutomatedODELAY_README.txt for specific instructions.
ODELAY_Cell Profiler_output.zip (1.3 Gb)
Output files from ODELAY_CellProfiler_pipeline.cp using the sample data set. Each folder is strain specific and contains and image summary file, object measurement file and overlay images displaying detected object boundaries at each time point.
ODELAY_MATLAB_input.zip (312 kb)
Folder containing ODELAY MATLAB tab-delimited text input files for the provided sample data set, named in the standard format (Cell ProfilerOUT_strain.txt).
ODELAY_MATLAB_pipeline.zip (48 kb)
This current version of the ODELAY MATLAB pipeline is described in Methods online. Refer to AutomatedODELAY_README.txt for specific instructions.
ODELAY_MATLAB_output.zip (160 kb)
Folder containing tab-delimited ODELAY MATLAB output text files for each strain in the provided sample data set. Refer to AutomatedODELAY_README.txt for a description of headings.

1. A method for determining at least one growth parameter of cells, said parameter selected from lag time, doubling time, carrying capacity and viability comprising:
a) spotting or pinning a plurality of spots each containing 1-5 up to several thousand cells in known separated spatial positions onto a spatially unimpeded solid growth medium on a transparent support;
b) allowing the cells in each spot to grow into a microcolony;
c) obtaining periodic simultaneous images of said microcolonies by high resolution optical detection; and
d) analyzing the images of said microcolonies within each of said spot to determine the size of said microcolonies as a function of time, to determine said growth parameter(s).

2. The method of claim 1, wherein said plurality includes at least 10 spots.

3. The method of claim 2, wherein at least 10 periodic images are obtained.

4. The method of claim 3, wherein the periodic images are obtained at intervals of 10 minutes-24 hours.

5. The method of claim 1, wherein the size of said microcolonies is calculated from the cross-sectional area of microcolonies for relative growth rates and/or three dimensionally modeled to determine volume from cross-sectional area to determine absolute doubling times.

6. The method of claim 5, wherein the cells are yeast and the size of the microcolonies is calculated from the cross-sectional area modeled to the base of a hemisphere.

7. The method of claim 1, wherein the periodic images are obtained during exponential growth stage of the cells, or
wherein the periodic images are obtained during the lag phase of the cells, or
wherein the periodic images are obtained during the stationary stage of the cells.

8. (canceled)

9. (canceled)

10. The method of claim 1, wherein the cells are yeast, bacteria, protozoa, fungi, archaea, or cells derived from a multicellular organism.

11. The method of claim 10, wherein the cells are Saccharomyces cerevisiae, Halobacterium salinarum, Synechococcus elongatus or Mycobacterium tuberculosis.

12. The method of claim 1, wherein the cells are labeled.

13. The method of claim 12, wherein the label comprises an optically-determinable dye or a multiplexed color combination.

14. The method of claim 13, wherein the label comprises a fluorescent moiety and/or a quantum dot.

15. The method of claim 1, wherein the growth medium is different among said spatial positions.

16. The method of claim 1, wherein the growth medium comprises magnetic particles in a predetermined arrangement on the surface of the medium to allow accurate automatic focusing.

17. The method of claim 1, wherein the transparent support consists of glass, transparent polymer resin or quartz.

18. The method of claim 1, wherein high resolution optical detection is by a digital camera.

19. The method of claim 1, which further includes analyzing the shape of the microcolonies wherein the shape is correlated with phenotype.

20. The method of claim 14, wherein said fluorescent moiety is coupled to a reporter protein for gene expression, and wherein said method further includes analyzing the fluorescence intensity as a measure of gene expression.

21. The method of claim 1, which includes assessing at least one growth parameter among the plurality of spots representing cells from a single sample.

22. A method for determining gene expression as a function of time in cells that have been modified to express a reporter product of said gene which method comprises:
a) spotting or pinning a plurality of spots, wherein each spot contains from 1-5 up to thousands of seeded cells in known separated spatial positions onto a spatially unimpeded solid growth medium on a transparent support;
b) allowing the cells in each spot to grow into a microcolony, where each microcolony develops from a single cell (or small group of seeded cells);
c) obtaining periodic simultaneous images of said microcolonies by high resolution optical detection; and
d) analyzing the images of said microcolonies within each of said spot to determine the level of reporter product as a function of time, to determine said gene expression.

23. The method of claim 22, wherein said reporter product is fluorescently labeled.

24. The method of claim 22, wherein the cells are yeast, bacteria, protozoa, fungi, archaea, or cells derived from a multicellular organism.

25. The method of claim 22, wherein the growth medium is different among said spatial positions.

26. The method of claim 22, wherein the growth medium comprises magnetic particles in a predetermined arrangement on the surface of the medium to allow accurate automatic focusing.

27. The method of claim 22, which further includes assessing variability in at least one growth parameter among the plurality of spots representing cells from a single sample.

28. The method
of claim 1 which further includes conducting said method in the presence and absence of said compound or composition; and
comparing the parameter(s) measured in the presence and absence of said compound or composition;
whereby a difference in said parameter(s) is indicative of an effect on said cell growth parameter(s) of the compound or composition.

29. The method of claim 28, wherein said compound or composition is a drug, a toxin, an environmental factor, or a growth factor.

30. A method for determining the effect of a test compound or composition on growth rate or cellular fitness which comprises:
conducting the method of claim 21 in the presence and absence of said compound or composition wherein said plurality of spots represents cells from a single sample, and
comparing the calculated growth parameters (lag time, doubling time, and carrying capacity) measured in the presence and absence of said compound or composition
wherein a difference in said growth parameters is indicative of an effect of the compound or composition on cellular fitness.

31. The method of claim 30, wherein said compound or composition is a drug, a toxin, an environmental factor, or a growth factor.

32. The method
of claim 22 which further includes conducting said method in the presence and absence of said compound or composition, and
comparing the levels of reporter product measured in the presence and absence of said compound or composition
whereby a difference in said levels is indicative of an effect of the compound or composition on gene expression.

33. The method of claim 32, wherein said compound or composition is a drug, a toxin, an environmental factor, or a growth factor.
